(19) United States
(12) Patent Application Publication
(10) Pub. No.: US 2010/0028924 A1
(43) Pub. Date: Feb. 4, 2010

(54) Method Of Diagnosing Pon1-Hdl Associated Lipid Disorders

(75) Inventors: Leonid Gaydukov, Moscow (RU); Olga Khersonsky, Jerusalem (IL); Dan S. Tawfik, Zur-Hadassa (IL); Michael Aviram, Haifa (IL)

Correspondence Address: MARTIN D. MOYNIHAN d/b/a PRTSI, INC., P.O. BOX 16446, ARLINGTON, VA, 22215, US

(73) Assignee: Rappaport Family Institute Research in the Medical Sciences, Haifa (IL)

(21) Appl. No.: 12/225,112

(22) PCT Filed: Mar. 18, 2007
(86) PCT No.: PCT/IL07/00345

§ 371 Date: Sep. 17, 2009

Related U.S. Application Data
(60) Provisional application No. 60/782,514, filed on Mar. 16, 2006.

(51) Int. Cl. C12Q 1/44

(52) U.S. Cl. 435/19

(57) ABSTRACT

Methods and kits for diagnosing a lipid-related disorder are disclosed. Methods and pharmaceutical compositions for treating lipid-related disorders are also disclosed.

### FIELD AND BACKGROUND OF THE INVENTION

The present invention relates to methods of diagnosing and treating PON1-lipoprotein (e.g., HDL) associated lipid disorders.

Atherosclerosis is a disorder characterized by cellular changes in the arterial intima and the formation of arterial plaques containing intracellular and extracellular deposits of lipids. The thickening of artery walls and the narrowing of the arterial lumen underlies the pathologic condition in most cases of coronary artery disease, aortic aneurysm, peripheral vascular disease, and stroke. A number of metabolic pathways and a cascade of molecular events are involved in the cellular morphogenesis, proliferation, and cellular migration that results in atherogenesis (Libby et al. (1997) Int J Cardiol 62 (S2):23-29).

Serum paraoxonase (PON1) is an HDL-associated enzyme playing an important role in the prevention of atherosclerosis. Serum PON1 levels and catalytic proficiency are inversely related to the risk of coronary heart disease [Aviram, M., Mol Med Today, 1999. 5(9): p. 381-6; Mackness, B., et al., Circulation, 2003. 107(22): p. 2775-9], and PON1 knockout mice are highly susceptible to atherosclerosis [Shih, D. M., et al., Nature, 1998. 394(6690): p. 284-7]. HDL-bound PON1 can inhibit the oxidative modification of lipids in LDL [Aviram, M., et al., Arterioscler Thromb Vasc Biol, 1998. 18(10): p. 1617-24] and enhance cholesterol efflux from macrophages [Rosenblat, M., et al., Atherosclerosis, 2005. 179(1): p. 69-77]. Despite its key antiatherognic role, the structure and enzymology of PON1 have been resolved only recently. PON1 hydrolyzes a broad range of substrates and has been traditionally described as paraoxonase/arylesterase. However, it recently became apparent that PON1 is in fact a lactonase with lipophylic lactones comprising its primary substrates [Khersonsky, O. and D. S. Tawfik, Biochemistry, 2005. 44(16): p. 6371-82], and that the arylesterase and paraoxonase activities are merely promiscuous [Aharoni, A., et al., Nat Genet, 2005. 37(1): p. 73-6].

It has been shown that, HDL particles carrying apolipoprotein A-I (apoA-I) bind PON1 with high affinity (nM), and thereby dramatically stabilize the enzyme and stimulate its lipo-lactonase activity (whilst the promiscuous paraoxonase and arylesterase activities are barely affected) [Gaidukov, L. and D. S. Tawfik, Biochemistry, 2005. 44(35): p. 11843-11854]. It has been also shown that impairing the lactonase activity of PON1, through mutations of its catalytic dyad, diminishes PON1's ability to prevent LDL oxidation and stimulate macrophage cholesterol efflux, indicating that the anti-atherogenic functions of PON1 are likely to be mediated by its lipo-lactonase activity [Khersonsky, O. and D. S. Tawfik, J Biol Chem, 2006; Rosenblat, M., et al., J Biol Chem, 2006].

Human PON1 has two common polymorphic sites: L55M that results in some quantitative differences in enzyme concentration, and R192Q that accounts for altered substrate specificity of the two isozymes [Smolen, A., Eckerson, H. W., Gan, K. N., Hailat, N., and La Du, B. N. (1991) Drug Metab Dispos 19, 107-112]. The R allele exhibits several fold higher activity toward paraoxon, while the arylesterase activity and PON1 levels are similar for the two isozymes [Humbert, R., Adler, D. A., Disteche, C. M., Hassett, C., Omiecinski, C. J., and Furlong, C. E. (1993) Nat Genet 3, 73-76]. The two isozymes also hydrolyze a number of lactones at slightly different rates. The R/Q polymorphism accounts for the bimodial distribution of the paraoxonase activity of individual human serum samples. PON1 R isozyme was also found to undergo higher enzymatic stimulation by NaCl. These differences in isozymic properties allowed to phenotype serum samples by dividing the paraoxonase activity in the presence of 1 M NaCl by the arylesterase activity.

Numerous case control studies have been conducted in the attempt to relate PON1 R/Q polymorphism with the incidence of cardiovascular disease. These reports are conflicting however, with some studies showing that the RR genotype is more closely associated with cardiovascular disease, and others indicating association with neither alleles. More recent studies concluded that the genotype, as well as enzyme levels and activity are important variables. None of the previous studies, however, examined PON1 in light of it being an interfacially-activated lipo-lactonase. The assays used phenyl acetate or paraoxon that bear no physiological relevance.

There are many treatments for the management and prevention of atherosclerosis and associated disorders such as hypercholesterolemia. Such current in-use drugs include statins (inhibition of cholesterol biosynthesis); beta-blockers (reduce hypertension or pulse rate); aspirin (helps in prevention of inflammation or blood clotting); and anti-oxidants (prevention of LDL modification).

U.S. Pat. Appl. No. 20030027759 teaches a method of decreasing an atheroma by treating with PON1. U.S. Pat. Appl. No. 20030027759 does not teach treating an atheroma with a particular PON1 isozyme.

Due to its high incidence and high variability in treatment response, there still remains a widely recognized need for, and it would be highly advantageous to have more effective indicators for diagnosing atherosclerosis as well as novel drugs for treating atherosclerosis and related diseases characterized by improved therapeutic activity.

### SUMMARY OF THE INVENTION

According to one aspect of the present invention there is provided a method of determining a stability of a serum PON1-lipoprotein complex, the method comprising measuring an inactivation rate of an enzymatic activity of a PON1 of the PON1-lipoprotein complex, thereby determining the stability of the serum PON1-lipoprotein complex.

According to another aspect of the present invention there is provided a method of determining an amount of a stable serum PON1-lipoprotein complex, the method comprising: (a) determining a fraction of stable serum PON1-lipoprotein complex: total serum PON1-lipoprotein complex, wherein inactivation of a stable complex follows the kinetics of a second phase of a double-exponential inactivation plot; and (b) determining a total level of serum PON1, wherein the fraction multiplied by the total level of serum PON1 is the amount of stable serum PON1-HDL apoA-I complex.

According to yet another aspect of the present invention there is provided a method of determining an amount of a stable serum PON1-lipoprotein complex, the method comprising: (a) determining a fraction of stable serum PON1-lipoprotein complex: total serum PON1-lipoprotein complex, following inactivation with an inactivator for a predetermined time at 37° C., wherein inactivation of a stable complex follows the kinetics of a second phase of a double-exponential inactivation plot; and (b) determining a total level of serum PON1, wherein the fraction multiplied by the total level of serum PON1 is the amount of stable serum PON1-HDL apoA-I complex.

According to still another aspect of the present invention there is provided a method of determining a normalized lactonase activity of serum PON1, the method comprising determining in a sample of a subject: (a) a lactonase activity of serum PON1; and (b) a total level of serum PON1, whereby a ratio of the lactonase activity: the total level is the normalized lactonase activity of serum PON1.

According to an additional aspect of the present invention there is provided a method of diagnosing a lipid-related disorder, the method comprising determining in a sample of a subject a normalized lactonase activity of PON1, thereby diagnosing the lipid-related disorder.

According to yet an additional aspect of the present invention there is provided a method of diagnosing a lipid-related disorder, the method comprising determining in a sample of a subject a fraction of serum PON1-lipoprotein complex: total PON1-lipoprotein complex, thereby diagnosing the lipid-related disorder.

According to still an additional aspect of the present invention there is provided a kit for determining a stability of a serum PON1-lipoprotein complex, the kit comprising at least one agent for measuring an inactivation rate of an enzymatic activity of a PON1 of said PON1-lipoprotein complex.

According to a further aspect of the present invention there is provided a kit for diagnosing a lipid-related disorder, the kit comprising at least one agent for determining in a sample of a subject a fraction of stable serum PON1: HDL apoA-I complex.

According to yet a further aspect of the present invention there is provided a kit for diagnosing a lipid-related disorder, the kit comprising at least one agent for determining a normalized lactonase activity of PON1 and instructions for measuring a normalized lactonase activity of PON1.

According to still a further aspect of the present invention there is provided a pharmaceutical composition comprising as an active ingredient an agent for increasing expression of an arginine-containing polymorph at position 192 of a PON1 polypeptide as set forth in SEQ ID NO: 14 and a pharmaceutically acceptable carrier.

According to still a further aspect of the present invention there is provided a method of treating a lipid-related disorder, comprising administering to a subject in need thereof a therapeutically effective amount of the pharmaceutical composition of the present invention, thereby treating the lipid-related disorder.

According to further features in preferred embodiments of the invention described below, the PON1-lipoprotein complex comprises HDL-apoA-I.

According to further features in preferred embodiments of the invention described below, the lipid-related disorder is selected from the group consisting of a cardiovascular disorder, a pancreatic disorder and a neurological disorder.

According to still further features in the described preferred embodiments, the cardiovascular disorder is selected from the group consisting of atherosclerosis, coronary heart disease, myocardial infarction, peripheral vascular diseases, venous thromboembolism and pulmonary embolism.

According to still further features in the described preferred embodiments, the cardiovascular disorder is not stroke.

According to still further features in the described preferred embodiments, the pancreatic disorder is type I or type II diabetes.

According to still further features in the described preferred embodiments, the method further comprises determining a presence or an absence, in a homozygous or a heterozygous form of an adenine-containing allele at position 575 of PON1 polynucleotide as set forth in SEQ ID NO: 15; and/or a glutamine-containing polymorph at position 192 of a PON1 polypeptide as set forth in SEQ ID NO:16.

According to still further features in the described preferred embodiments, the kit further comprises at least one agent for determining a presence or an absence, in a homozygous or a heterozygous form of an adenine-containing allele at position 575 of PON1 polynucleotide as set forth in SEQ ID NO: 15; and/or a glutamine-containing polymorph at position 192 of a PON1 polypeptide as set forth in SEQ ID NO:16.

According to still further features in the described preferred embodiments, the determining the presence or absence of said adenine-containing allele at position 575 of said PON1 polynucleotide is effected by a method selected from the group consisting of: DNA sequencing, restriction fragment length polymorphism (RFLP analysis), allele specific oligonucleotide (ASO) analysis, Denaturing/Temperature Gradient Gel Electrophoresis (DGGE/TGGE), Single-Strand Conformation Polymorphism (SSCP) analysis, Dideoxy fingerprinting (ddF), pyrosequencing analysis, acycloprime analysis, Reverse dot blot, GeneChip microarrays, Dynamic allele-specific hybridization (DASH), Peptide nucleic acid (PNA) and locked nucleic acids (LNA) probes, TaqMan, Molecular Beacons, Intercalating dye, FRET primers, AlphaScreen, SNPstream, genetic bit analysis (GBA), Multiplex minisequencing, SNaPshot, MassEXTEND, MassArray, GOOD assay, Microarray miniseq, arrayed primer extension (APEX), Microarray primer extension, Tag arrays, Coded microspheres, Template-directed incorporation (TDI), fluorescence polarization, Colorimetric oligonucleotide ligation assay (OLA), Sequence-coded OLA, Microarray ligation, Ligase chain reaction, Padlock probes, Rolling circle amplification, and Invader assay.

According to still further features in the described preferred embodiments, the determining the presence or absence of the glutamine-containing polymorph at position 192 of a PON1 polypeptide is effected by an agent capable of binding to either the Q containing polymorph at position 192 of the PON1 polypeptide or an R containing polymorph at position 192 of the PON1 polypeptide.

According to still further features in the described preferred embodiments, the agent is an antibody capable of binding the Q containing polymorph at position 192 of the PON1 polypeptide and not binding an R containing polymorph at position 192 of the PON1 polypeptide.

According to still further features in the described preferred embodiments, the agent is an antibody capable of binding the R containing polymorph at position 192 of the PON1 polypeptide and not binding an Q containing polymorph at position 192 of the PON1 polypeptide.

According to still further features in the described preferred embodiments, the method further comprises determining a lactonase activity of serum PON1.

According to still further features in the described preferred embodiments, the method further comprises determining in a sample of the subject a fraction of stable PON1-lipoprotein complex: total PON1-lipoprotein complex.

According to still further features in the described preferred embodiments, the method further comprises determining in a sample of the subject an amount of total PON1-lipoprotein complex.

According to still further features in the described preferred embodiments, the kit further comprises at least one agent for determining a lactonase activity of serum PON1.

According to still further features in the described preferred embodiments, the kit further comprises at least one agent for determining an amount of total serum PON1.

According to still further features in the described preferred embodiments, the lactonase activity is a normalized lactonase activity.

According to still further features in the described preferred embodiments, the kit further comprises at least one agent for determining in a sample of a subject a fraction of stable serum PON1-lipoprotein complex: total PON1-lipoprotein complex.

According to still further features in the described preferred embodiments, the determining the lactonase activity of serum PON1 is effected using 5-(thiobutyl)-butyrolactone (TBBL).

According to still further features in the described preferred embodiments, the at least one agent is 5-(thiobutyl)-butyrolactone (TBBL).

According to still further features in the described preferred embodiments, the determining an amount of a stable serum PON1-lipoprotein complex is effected by: (a) determining a fraction of stable serum PON1-lipoprotein complex: total serum PON1-lipoprotein complex; and (b) determining a total level of serum PON1, wherein the fraction multiplied by the total level of serum PON1 is the amount of stable serum PON1-HDL apoA-I complex.

According to still further features in the described preferred embodiments, the determining a fraction of stable serum PON1-lipoprotein complex is effected by measuring an inactivation rate of an enzymatic activity of a PON1 of the PON1-HDL apoA-1 complex.

According to still further features in the described preferred embodiments, the measuring an inactivation rate is effected using a PON1 inactivator.

According to still further features in the described preferred embodiments, the at least one agent for determining the fraction of stable serum PON1-lipoprotein complex is an agent capable of measuring an inactivation rate of an enzymatic activity of a PON1 of the PON1-lipoprotein complex.

According to still further features in the described preferred embodiments, the agent is a PON1 inactivator.

According to still further features in the described preferred embodiments, the kit further comprises phenyl acetate.

According to still further features in the described preferred embodiments, the PON1 inactivator is NTA.

According to still further features in the described preferred embodiments, the agent is a polypeptide as set forth in SEQ ID NO: 14.

According to still further features in the described preferred embodiments, the agent is a polynucleotide as set forth in SEQ ID NO: 13.

According to still further features in the described preferred embodiments, the agent is an oligonucleotide.

The present invention successfully addresses the shortcomings of the presently known configurations by providing methods and kits for diagnosing lipid-related disorders.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a graph showing the inactivation kinetics of the wild-type rePON1-192K (SEQ ID NO:2), -192R (SEQ ID NO:4) and -192Q (SEQ ID NO:6) isozymes, bound to rHDL-apoA-I, or in buffer. Delipidated enzymes were incubated with a 50-fold excess of rHDL-apoA-I, or in activity buffer, and subjected to inactivation in the presence of EDTA (5 mM) and β-mercaptoethanol (10 mM) at 37° C. Residual activity at various time points was determined by initial rates of phenyl acetate hydrolysis (2 mM) and plotted as percent of the activity at time zero. Data were fitted to a single exponential for wild-type rePON1-192K on rHDL-apoA-I, and to double-exponentials for the remaining samples. The inactivation rate constants and amplitudes were derived from this fit;

FIGS. 2A-C are sensorgrams for the binding of wt Δ20-rePON1-192K—SEQ ID NO:8 (FIG. 2A), Δ20-rePON1-192R—SEQ ID NO:10 (FIG. 2B) and Δ20-rePON1-192Q—SEQ ID NO:12 (FIG. 2C) isozymes to rHDL-apoA-I. Biotinylated rHDL-apoA-I particles were immobilized onto the streptavidin surface (SA chip), and a series of PON1 concentrations were injected over the immobilized and blank surfaces to obtain the net binding response. Binding was performed at 25° C. Association and dissociation phases were fitted to a single exponential, from which kinetic rate constants were derived. PON1 concentrations in all the sensorgrams are (from bottom to top) 25, 40, 60, 80, 100, 150 and 200 nM;

FIGS. 3A-B are graphs showing stimulation of the lipo-lactonase activity of the rePON1 isozymes by rHDL-apoA-I. Delipidated enzymes (0.2 μM) were incubated with increasing concentrations of rHDL-apoA-I, and enzymatic activity was determined with δ-nonanoic lactone (1 mM) (FIG. 3A) and TBBL (0.25 mM) (FIG. 3B). The activity is presented in relation to the initial activity of the delipidated enzymes (percentage of stimulation). Data were fitted to the Langmuir saturation curve, from which the activation factor (Vₘₐₓ) and the apparent affinity (Kₐₚₚ) were derived;

FIGS. 4A-D are graphs showing stimulation of the lactonase activity (FIGS. 4A-B) and promiscuous esterase and phosphotriesterase activities (FIGS. 4C-D) of rePON1 isozymes by rHDL-apoA-I. Delipidated enzymes were incubated with increasing concentrations of rHDLs (rHDL/rePON molar ratios of 0.5-50), and enzymatic activity was determined with γ-dodecanoic lactone (FIG. 4A), δ-valerolactone (FIG. 4B), phenyl acetate (FIG. 4C), and paraoxon (FIG. 4D) (each substrate at 1 mM). Stimulated activity is presented as the percentage of the initial activity of the delipidated enzymes (corresponds to 100%). Data were fitted to the Langmuir saturation curve, from which the activation factor (Vₘₐₓ) and the apparent affinity (Kₐₚₚ) were derived;

FIGS. 5A-B are graphs showing the inactivation and catalytic stimulation of the human PON1 192R/Q isozymes. FIG. 5A is a line graph showing the inactivation kinetics of human PON1-192R and -192Q in the presence of rHDL-apoA-I. The delipidated isozymes (0.2 μM) were incubated with a 50-fold molar excess of rHDL-apoA-I in activity buffer, and subjected to inactivation by NTA and β-mercaptoethanol (each at 5 mM) at 25° C. Residual activity determined as above (FIG. 1) and data were fitted to a double-exponential function. FIG. 5B is a bar graph showing stimulation of the enzymatic activity of human PON1-192R and -192Q by rHDL-apoA-I. The delipidated isozymes were incubated with a 50-fold molar excess of rHDL-apoA-I, and enzymatic activity was determined with various substrates (at 1 mM for all the substrates, except TBBL at 0.25 mM). The activity is presented in relation to the initial activity of the delipidated enzymes (percentage of stimulation). Each bar represents the mean and S.D. of at least two independent measurements;

FIG. 6 is a scatter plot showing PON1 192R/Q phenotyping of human sera from 54 healthy individuals. Sera were phenotyped using the conventional two-substrate method by measuring the ratio of the paraoxonase to aryl esterase activity and the percentage of the paraoxonase activity stimulation by 1 M NaCl. Paraoxonase to phenyl acetate ratios for QQ, RQ and RR sera types were <3, 3-7 and >7, respectively, and the percentages of activity stimulation by NaCl were <70, 70-150, and >150%, respectively. Out of 54 sera, 34 were phenotyped as QQ, 14 as RQ and 6 as RR;

FIGS. 7A-B are graphs showing PON1 inactivation assays with human sera. FIG. 7A is a line graph showing the kinetics of PON1 inactivation in 16 selected human sera of QQ, RQ and RR phenotype (PON1 phenotyping was performed by the two-substrate method as described in the Methods section). Human sera from healthy individuals were diluted 10-fold in TBS (50 mM Tris pH 8.0, 150 mM NaCl) and subjected to inactivation by 0.25 mM NTA and 1 mM β-mercaptoethanol at 25° C. Residual activity was determined by initial rates of phenyl acetate hydrolysis (2 mM) and plotted as percent of the rate at time zero. Data were fitted to mono-exponentials for RR-type sera, and double-exponentials for RQ- and QQ-type sera. FIG. 7B is a scatter plot showing PON1 stability, expressed as the percent residual activity following 9 hrs of inactivation, for 54 samples of human sera belonging to the QQ, RQ and RR phenotypes. Horizontal bars represent the mean stability of each group;

FIG. 8 is a scatter plot showing the correlation between PON1 stability (referred as percentage residual activity following 9 hrs of inactivation) and enzyme levels (expressed as dihydrocoumarin activity) in human sera. PON1 levels were measured with dihydrocoumarin (1 mM) and expressed in Units/mL (1 μmol of dihydrocoumarin hydrolyzed per min per 1 ml serum).

Positive correlation was found for Q-type and RQ-type sera (correlation factor for linear regression=1.2 and 0.5, respectively; R=0.54 and 0.35, respectively);

FIGS. 9A-D are scatter plots showing lactonase activity, PON1 levels, lactonase stimulation and fraction of tightly HDL-bound PON1 in human sera taken from 54 healthy individuals belonging to the QQ, RQ and RR phenotypes. Horizontal bars represent the mean value for each group. FIG. 9A illustrates levels of lactonase activity measured with TBBL (0.25 mM) and expressed in Units/mL (1 μmol of TBBL hydrolyzed per min per 1 ml serum). FIG. 9B illustrates levels of activity with dihydrocoumarin (DHC, 1 mM) expressed in Units/mL (1 μmol of dihydrocoumarin hydrolyzed per min per 1 ml serum). FIG. 9C illustrates the ‘normalized’ lactonase activity expressed as the ratio of TBBLase to dihydrocoumarin activity for each sample. FIG. 9D is an amplitude of the slow phase of inactivation (A₂, %) that was derived from inactivation assay (FIG. 7A), and corresponds to the fraction of tightly HDL-bound PON1.

FIG. 10 is an estimation of the levels of PON1-HDL complex. These levels (in arbitrary units) were obtained by multiplying, for each serum sample, the amplitude of the slow phase of inactivation (A₂) which corresponds to the fraction of “tightly” HDL-bound PON1 by the levels of dihydrocoumarin activity which correspond to the total concentration of serum PON1. Since the ratio of Vₘₐₓ of PON1-192Q and -192R isozymes toward dihydrocoumarin is 1.125, the levels of dihydrocoumarin activity of the RR and RQ sera were corrected by factors of 1.125, and 1.0625, respectively;

FIG. 11A-B are graphs showing PON1 inactivation assays with human sera. FIG. 11A is a line graph showing the kinetics of PON1 inactivation in 7 selected human sera of QQ, RQ and RR phenotype (PON1 phenotyping was performed by the two-substrate method as described in FIG. 6). Human sera were diluted 10-fold in TBS (50 mM Tris pH 8.0, 150 mM NaCl) and subjected to inactivation by 2 mM NTA and 10 mM β-mercaptoethanol at 37° C. Residual activity was determined by initial rates of phenyl acetate hydrolysis (2 mM) and plotted as percent of the rate at time zero. Data were fitted to double-exponentials, from which the kinetic rate constants and the amplitudes of inactivation phases were determined. FIG. 7B is a linear correlation fit between the percent residual PON1 activity following 2 hrs of inactivation and the amplitude of the slow phase of inactivation (A₂) which corresponds to the fraction of “tightly” HDL-bound PON1. Correlation coefficient for linear regression (R)=0.99.

FIG. 12 is a bar graph showing cholesterol efflux rates in the presence of the HDL-bound rePON1 isozymes. The wild-type rePON1-192K and its -192R and -192Q isozymes were incubated with 2.5 or 5-fold excess of rHDL-apoA-I, and added (at the final concentration of 0.4 μM rePON1 and 1 μM rHDL) to the cultured macrophages pre-incubated with [³H]-labeled cholesterol. The degree of HDL-mediated cholesterol efflux was calculated by measuring the cellular and medium [³H]-label after 3 hrs incubation at 37° C. Each bar represents the mean and SD of three measurements; and

FIGS. 13A-B are graphs showing PON1 stability (FIG. 13A) and normalized lactonase activity (FIG. 13B) in human sera from 54 healthy individuals. Sera phenotypes were determined using two-substrate method. PON1 stability in sera was determined by inactivation assay and expressed as the percentage residual activity following 9 hrs of inactivation. Normalized lactonase activity was determined by measuring the ratio of TBBL to dihydrocoumarin activity. Stability and normalized lactonase activity were plotted against the percentage of paraoxonase activity stimulation determined by the conventional phenotyping method (See FIG. 6).

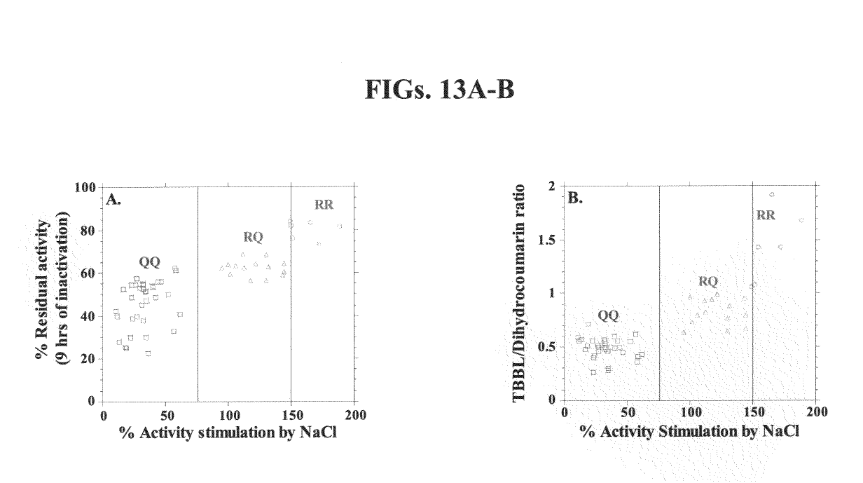

### DESCRIPTION OF THE PREFERRED EMBODIMENTS

The present invention relates to methods and kits for determining the predisposition of an individual to a lipid-related disorder and pharmaceutical compositions and methods of treating same.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

PON1 is a lipoprotein (HDL)-associated enzyme with anti-atherogenic and detoxification properties that hydrolyzes a wide range of substrates, such as esters, organophosphates (e.g., paraoxon) and lactones.

For a long time, PON1 was considered an aryl-esterase and paraoxonase, and its activity was measured accordingly. However, it recently became apparent that PON1 is primarily a lactonase catalyzing both the hydrolysis and formation of a variety of lactones. In addition, it was found that PON1 is an interfacially-activated lactonase that selectively binds apoA-I containing HDL, and is thereby greatly stabilized (>100-fold) and catalytically activated (>10-fold) towards lipophylic lactones.

HDL-bound PON1 inhibits LDL oxidation and stimulates cholesterol efflux from macrophages. PON1 knockout mice are highly susceptible to atherosclerosis. Accordingly, serum PON1 levels seem to be inversely related to the level of cardiovascular disease, although this correlation is week. Impairing the lactonase activity of PON1, through mutations of its catalytic dyad, diminishes PON1's ability to prevent LDL oxidation and stimulate macrophage cholesterol efflux, indicating that the anti-atherogenic functions of PON1 are likely to be mediated by its lipo-lactonase activity. Accordingly, in term of atherosclerosis, the HDL-bound PON1 (or other lipoproteins as is described further below) represents the biologically relevant portion of PON1.

Prior art methods used to correlate PON1 with lipid-related disorders test overall PON1 level and promiscuous, non physiological activities (namely, arylesterase and phosphotriesterase) without taking into account the degree to which PON1 binds to HDL and thus the level of the HDL-PON1 complex and “relevant” PON1 activity (namely, the lactonase activity).

For example, U.S. Pat. Appl. No. 20030027759 teaches a method of diagnosing predisposition to hypercholesterolemia by assessing the level only of native circulating PON-1 in a mammal. U.S. Pat. Appl. No. 20030027759 also teaches a method of decreasing an atheroma by treating with PON1. U.S. Pat. Appl. No. 20030027759 does not teach treating an atheroma with a particular PON1 isozyme.

There is thus a need for methods of diagnosing lipid-related disorders that take into account the physiological activity (namely, the lipo-lactonase) as well as the levels of PON1, and the levels of the HDL-PON1 complex.

The present invention is based on the experimental evidence described herein below that assaying PON1's chelator-mediated inactivation rate may be used as an accurate gauge of HDL binding and thus of an amount of “atherosclerosis-relevant” PON-1 (namely, PON1 that is “tightly” or efficiently bound to HDL versus the loosely or non-efficiently bound enzyme). This new test, either alone or in combination with other parameters such as measurement of total PON1 levels and lipo-lactonase activity and PON1 genotyping is likely to be a better indicator, and possibly predictor, of atherosclerosis.

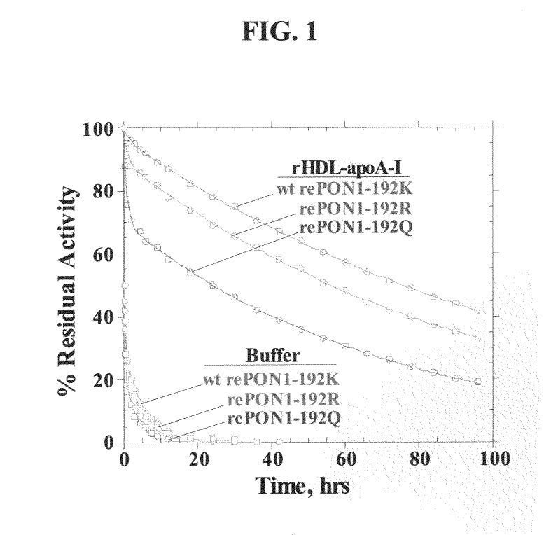

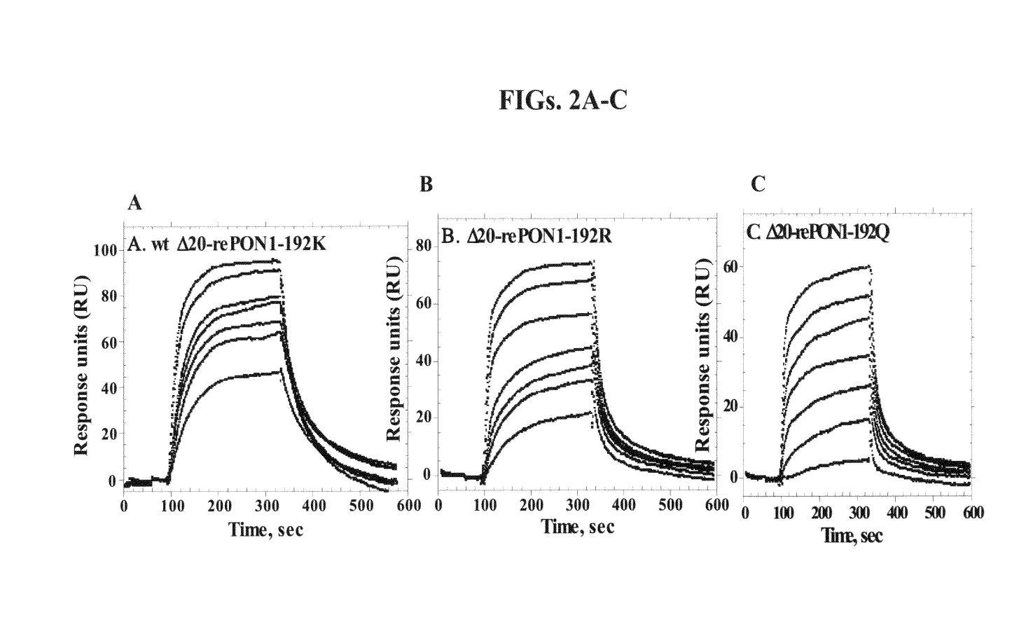

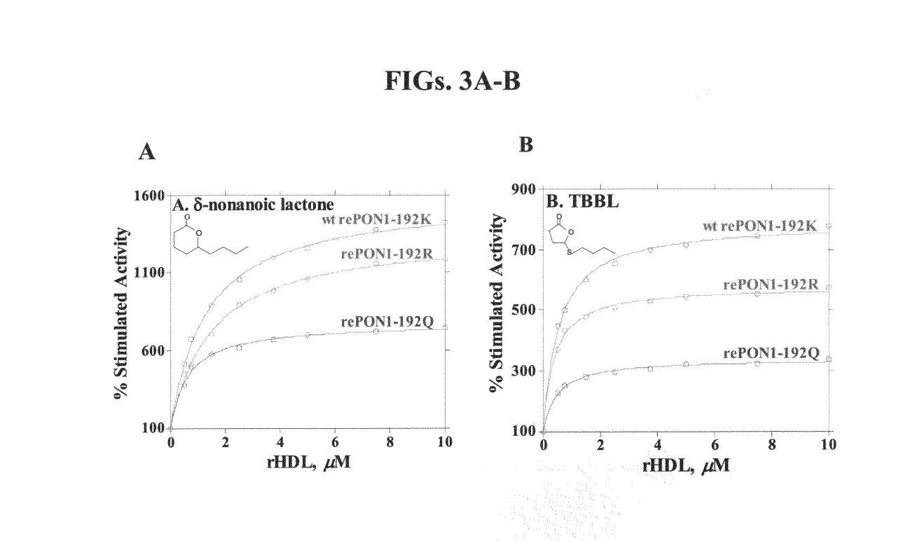

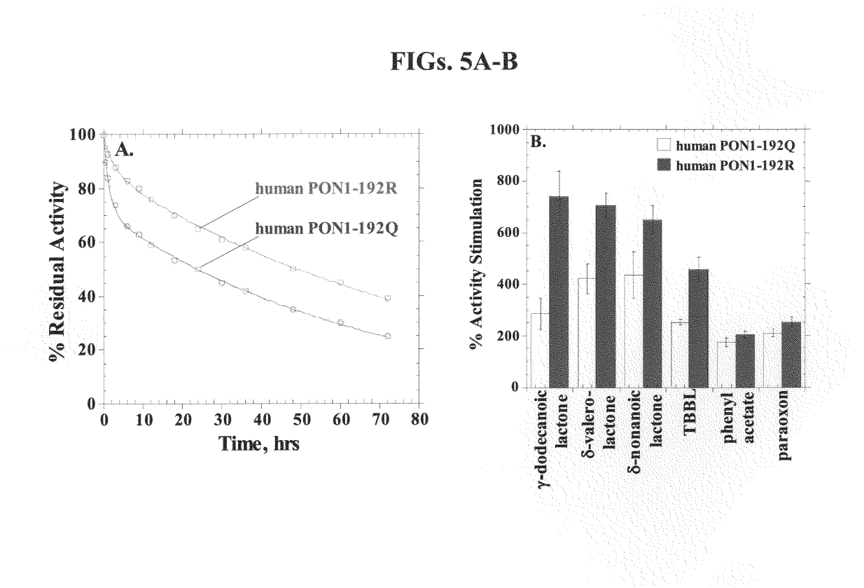

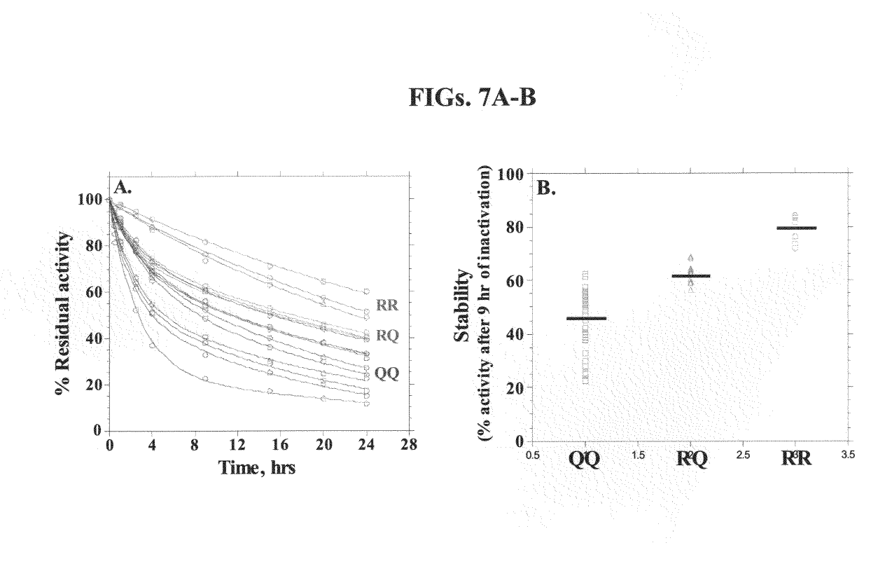

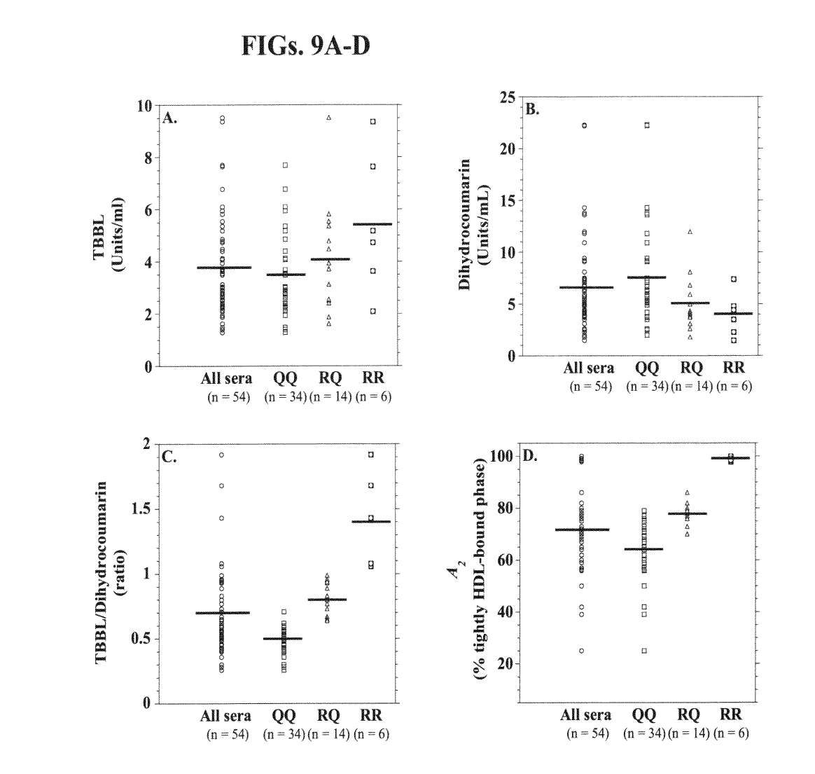

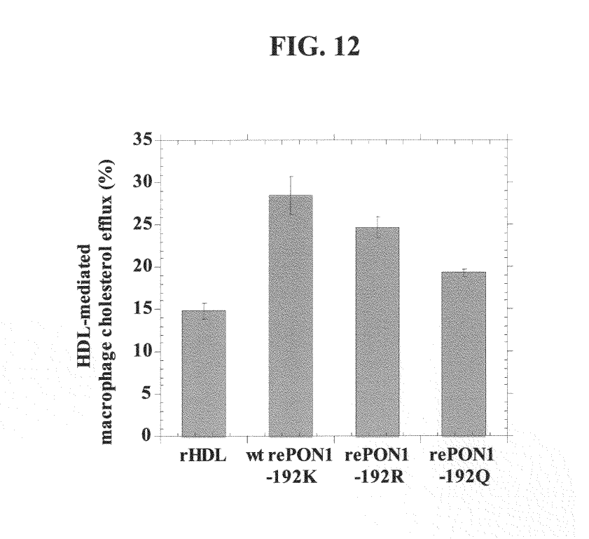

Furthermore, the results presented herein, unambiguously indicate that the PON1 R/Q isozymes differ in their HDL binding properties and as a result, in their stability and lipo-lactonase activity. Thus the R isozyme binds HDL with a higher affinity as measured by inactivation kinetics (FIG. 1) and by surface plasmon resonance (SPR) measurements (FIGS. 2A-C). Consequently, the R isozyme exhibits much higher stability (FIG. 1 and FIG. 5A) and lactonase activity when bound on HDL-apoA-I (FIGS. 3A-B and 4A-D), as well as more potent antiatherogenic activity (FIG. 12). These differences in HDL-binding, stability and lactonase activity are also clearly observed in sera samples obtained from individuals belonging to the QQ, QR and RR genotypes. The three phenotypes of human sera (QQ, RQ and RR) exhibit different stabilities (FIG. 5A; FIGS. 7 A-B) and lactonase levels (FIGS. 9 A and 9C) at the expected order.

Thus, according to one aspect of the present invention there is provided a method of diagnosing a lipid-related disorder, the method comprising determining in a sample of a subject a fraction of stable serum PON1-lipoprotein complex (by determining the level of PON1 that is “tightly” bound to the lipoprotein): total serum PON1-lipoprotein complex, thereby diagnosing the lipid-related disorder.

The term “lipoprotein complex” refers to a complex between PON1 and a lipoprotein. Exemplary lipoproteins include HDL and VLDL. The lipoprotein complex may comprise apolipoproteins including, but not limited to apoA-I, apoA-II, apoE, apoA-IV and apoC.

The phrase “lipid related disorder” as used herein refers to a disorder which results from or associated with improper lipoprotein (e.g. HDL) activity. Without being bound by theory, it is suggested that lipoproteins such as HDL particles carrying apolipoprotein A-I (apoA-I) bind PON1 with high affinity (nM), and thereby dramatically stabilize the enzyme and stimulate its lipo-lactonase activity, resulting in an increased ability of PON1 to prevent LDL oxidation and stimulate macrophage cholesterol efflux. Examples of lipid-related disorders associated with altered HDL activities are described herein below.

As used herein, the term “diagnosing” refers to classifying a disease or a symptom as a lipid-related disorder, determining a predisposition to a lipid related disorder, determining a severity of a lipid related disorder, monitoring disease progression, forecasting an outcome of a disease and/or prospects of recovery.

According to this aspect of the present invention, a fraction of stable (i.e. tightly bound complex) out of the total complex below a predetermined threshold (“low level”) is indicative of a predisposition to and/or a condition associated with a lipid-related disorder.

Determination of accurate thresholds may be effected by measuring the fraction of stable complex in a statistically relevant group of individuals with lipid related disorders and comparing the fractions with those measured in statistically relevant groups of healthy individuals.

As used herein, “a stable complex” refers to a complex which following inactivation with an inhibitor follows the slow rate inactivation kinetics (inactivation rate of a second phase of a double exponential fit).

As used herein, “a non-stable complex” refers to a complex which following inactivation with an inhibitor follows the kinetics of a first (fast) phase of a double exponential inactivation plot.

An exemplary method for determining a fraction of stable serum PON1-lipoprotein complex is described in Example 2 herein below. According to this method, the fraction of tightly bound:total PON1 complex (i.e. stable PON1-lipoprotein complex:total PON1-lipoprotein complex) is determined by measuring the rate of inactivation of the complex in the presence of an inactivator. Specifically, an individual's serum is contacted with a PON-1 protein inactivator (e.g. 0.25 mM NTA) and a PON1 substrate (e.g. phenyl acetate). The rate of inactivation may be determined by measuring the residual enzymatic activity for the added substrate over time. Preferably sera are supplemented with β-mercaptoethanol (e.g. 5 mM), and stored 4° C. for 12 hours prior to the experiment to avoid oxidation.

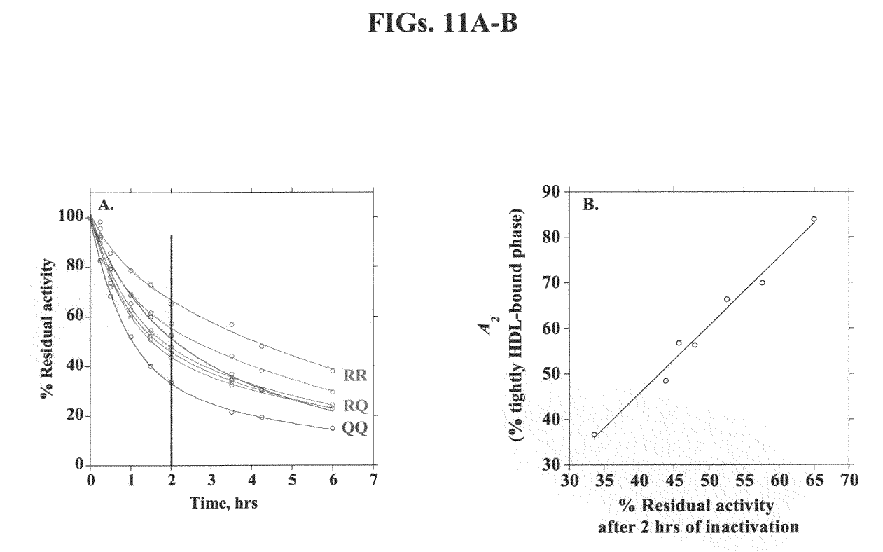

PON1 inactivation in serum can be also assayed using harsher conditions (e.g. a combination of 2 mM NTA and 10 mM β-mercaptoethanol at 37° C.) (see FIG. 11A). The fraction of tightly-bound PON1 (i.e., A₂, the slow phase of inactivation) can be derived from fitting the inactivation kinetics to the double-exponential function. Alternatively, the residual activity of PON1 in sera can be determined at a single fixed time point—i.e. a predetermined time point (e.g. following 2 hrs of inactivation). These values are found in excellent correlation with the A₂ values for different human sera (FIG. 11B).

Diagnosis of lipid related disorders may be effected by determining the percent (i.e. ratio) of stable complex alone or together with the total amount of PON1 levels. Multiplication of the ratio by the total amount provides an estimate of the total level of stable PON1:lipoprotein complex.

Determination of a total PON1 level may be effected by any method known in the art. For example, the level of total PON1 may be determined using an antibody specific for PON1. Preferably, the level of total PON1 is determined by measuring the level of total enzymatically active PON1. Thus for example, the level of total PON1 may be measured using an ELISA assay. Such assays are known in the art—see for example Blatter et al, Biochem J 304 (Pt 2): 549-554. An exemplary method for measuring total PON1's serum concentration comprises assaying PON1 dihydrocoumarin hydrolysis as described in Example 3. Unlike other lactones, dihydrocoumarin is not stimulated by HDL, and the PON1 isozymes hydrolyze it at nearly identical rates (at Vₘₐₓ^{Q}=1.125*Vₘₐₓ^{R}).

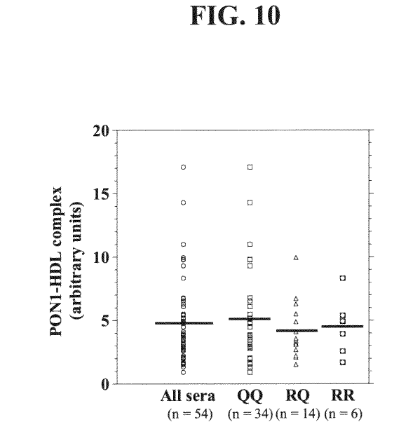

Further details relating to the calculation of the level of a stable PON1:lipoprotein complex can be found in the Examples section (See Figure legend for FIG. 10), herein above.

Since the stability of PON1:lipoprotein correlates with the affinity of PON1 for the lipoprotein (e.g. HDL), stability may also be measured by determining the affinity between the two. Methods of determining protein affinity are well known in the art [e.g., BiaCore and/or Scatchard analyses (RIA)]. Alternatively, methods such as surface plasmon resonance (SPR) may be used to measure protein affinity (see example 1 herein below).

Diagnosis of lipid related disorders may be made on the basis of the stability of the PON1 complex as described herein above, either alone or in conjunction with other diagnostic tests. According to a preferred embodiment, the lactonase activity of the complex and specific catalytic activity (i.e. lactonase activity as a function of total PON1 levels) are also measured so as to provide a more complete analysis of the PON1 status of an individual. Methods of measuring lactonase activity and specific catalytic activity are described herein below.

It will be appreciated that other tests may also be performed in conjunction with the stability testing of the present invention to obtain further evidence as to the HDL status and/or PON1 status of a subject. For example the diagnostic tests of the present invention may be performed in conjunction with other assays that address the levels of various types of HDLs, LDLs, apolipoproteins, and other proteins and factors related to atherosclerosis in order to comprise reliable indicators as well as predictors of atherosclerosis.

For example, the HDL status and/or PON1 status of a subject can be further analyzed by genotyping PON1 since the present inventors have shown that PON1 R/Q isozymes differ in their HDL binding properties and as a result, in their stability and lipo-lactonase activity.

Thus, another test which may be performed in conduction with the diagnostic test of the present invention is determining a presence or an absence, in a homozygous or a heterozygous form of an adenine-containing allele at position 575 of PON1 polynucleotide as set forth in SEQ ID NO: 15; and/or a glutamine-containing polymorph at position 192 of a PON1 polypeptide as set forth in SEQ ID NO:16.

As used herein the phrase “PON1 polynucleotide” refers to the DNA sequence on chromosome 7q21.3 of the human genome encoding PON1 enzyme as set forth by GenBank Accession No. NM_{—}000446 (version NM_{—}000446.3). As described in the Background section hereinabove, there are several genetic polymorphisms in the PON1 gene. One such polymorphism is the 192R/Q polymorphism which accounts for altered substrate specificity of the two isozymes. The R allele exhibits several fold higher activity toward paraoxon, while the arylesterase activity and PON1 levels are similar for the two isozymes. The two isozymes also hydrolyze a number of lactones at slightly different rates.

As used herein the phrase “PON1 polypeptide” refers to the polypeptide as set forth by GenBank Accession No. NP_{—}000437 (version NP_{—}000437.3).

The term “polymorphism” refers to the occurrence of two or more genetically determined variant forms (alleles) of a particular nucleic acid (or nucleic acids) of a nucleic acid sequence (e.g., gene) at a frequency where the rarer (or rarest) form could not be maintained by recurrent mutation alone. Polymorphisms can arise from deletions, insertions, duplications, inversions, substitution and the like of one or more nucleic acids. The polymorphism used by the present invention may be a single nucleotide polymorphism (SNP) which comprises the G/A substitution at position 575 of the PON1 gene. Such SNP is a non-synonymous polymorphism (i.e., results in an amino acid change in the translated protein) which comprises the R192Q substitution (i.e., a substitution of an arginine residue with a glutamine residue at position 192) of the PON1 polypeptide set forth by SEQ ID NO:16.

The terms “homozygous” or “heterozygous” refer to two identical or two different alleles and/or protein polymorphs, respectively, of a certain polymorphism.

The term “absence” as used herein with respect to the allele and/or the protein polymorph describes the negative result of a specific polymorphism determination test. For example, if the polymorphism determination test is suitable for the identification of an adenine nucleotide-containing allele at position 575 of the PON1 polynucleotide, and the individual on which the test is performed is homozygous for the guanine nucleotide-containing allele at position 575 of the PON1 polynucleotide, then the result of the test will be “absence of the adenine nucleotide—containing allele”. Similarly, if the polymorphism determination test is suitable for the identification of a glutamine residue—containing polymorph at position 192 of the PON1 polypeptide, and the individual on which the test is performed is homozygote for the arginine—containing polymorph at position 192 of the PON1 polypeptide, then the result of the test will be “absence of the glutamine residue—containing polymorph”.

Determining the presence or the absence of the PON1 575A allele according to this aspect of the present invention can be effected using a DNA sample which is derived from any suitable biological sample of the individual, including, but not limited to, blood, plasma, blood cells, saliva or cells derived by mouth wash, and body secretions such as urine and tears, and from biopsies, etc. Additionally or alternatively, nucleic acid tests can be performed on dry samples (e.g. hair or skin). In addition, the presence of PON1 192Q polymorph may be determined using a protein sample derived from serum. Methods of extracting DNA and protein samples from blood samples are well known in the art.

The PON1 575G/A SNP of the PON1 polynucleotide can be identified using a variety of approaches suitable for identifying sequence alterations. Following is a non-limiting list of SNP detection methods which can be used to identify the PON1 575G/A SNP of the present invention.

Restriction fragment length polymorphism (RFLP): This method uses a change in a single nucleotide (the SNP nucleotide) which modifies a recognition site for a restriction enzyme resulting in the creation or the destruction of an RFLP.

Single nucleotide mismatches in DNA heteroduplexes are also recognized and cleaved by some chemicals, providing an alternative strategy to detect single base substitutions, generically named the “Mismatch Chemical Cleavage” (MCC) (Gogos et al., Nucl. Acids Res., 18:6807-6817, 1990). However, this method requires the use of osmium tetroxide and piperidine, two highly noxious chemicals which are not suited for use in a clinical laboratory.

Allele specific oligonucleotide (ASO): In one embodiment, this method uses an allele-specific oligonucleotide (ASO) which is designed to hybridize in proximity to the polymorphic nucleotide, such that a primer extension or ligation event can be used as the indicator of a match or a mis-match. In another embodiment, the ASO is used as a hybridization probe, which due to the differences in the melting temperature of short DNA fragments differing by a single nucleotide, is capable of differentially hybridizing to a certain allele of the SNP and not to the other allele. It will be appreciated that stringent hybridization and washing conditions are preferably employed. Hybridization with radioactively labeled ASO also has been applied to the detection of specific SNPs (Conner et al., Proc. Natl. Acad. Sci., 80:278-282, 1983). Example of primers (end-labelled) that may be used according to this embodiment of the present invention are Gln-192 specific: 5′CCTACTTACAATCCTGGGA3′ and Arg-192 specific: 5′CCTACTTACGATCCTGGGA3′ [Humbert, R., Adler, D. A., Disteche, C. M., Hassett, C., Omiecinski, C. J., and Furlong, C. E. (1993) Nat Genet. 3, 73-76].

Denaturing/Temperature Gradient Gel Electrophoresis (DGGE/TGGE): Two other methods rely on detecting changes in electrophoretic mobility in response to minor sequence changes. One of these methods, termed “Denaturing Gradient Gel Electrophoresis” (DGGE) is based on the observation that slightly different sequences will display different patterns of local melting when electrophoretically resolved on a gradient gel. In this manner, variants can be distinguished, as differences in melting properties of homoduplexes versus heteroduplexes differing in a single nucleotide can detect the presence of SNPs in the target sequences because of the corresponding changes in their electrophoretic mobilities. The fragments to be analyzed, usually PCR products, are “clamped” at one end by a long stretch of G-C base pairs (30-80) to allow complete denaturation of the sequence of interest without complete dissociation of the strands. The attachment of a GC “clamp” to the DNA fragments increases the fraction of mutations that can be recognized by DGGE (Abrams et al., Genomics 7:463-475, 1990). Attaching a GC clamp to one primer is critical to ensure that the amplified sequence has a low dissociation temperature (Sheffield et al., Proc. Natl. Acad. Sci., 86:232-236, 1989; and Lerman and Silverstein, Meth. Enzymol., 155:482-501, 1987). Modifications of the technique have been developed, using temperature gradients (Wartell et al., Nucl. Acids Res., 18:2699-2701, 1990), and the method can be also applied to RNA:RNA duplexes (Smith et al., Genomics 3:217-223, 1988).

Limitations on the utility of DGGE include the requirement that the denaturing conditions must be optimized for each type of DNA to be tested. Furthermore, the method requires specialized equipment to prepare the gels and maintain the needed high temperatures during electrophoresis. The expense associated with the synthesis of the clamping tail on one oligonucleotide for each sequence to be tested is also a major consideration. In addition, long running times are required for DGGE. The long running time of DGGE was shortened in a modification of DGGE called constant denaturant gel electrophoresis (CDGE) (Borrensen et al., Proc. Natl. Acad. Sci. USA 88:8405, 1991). CDGE requires that gels be performed under different denaturant conditions in order to reach high efficiency for the detection of SNPs.

A technique analogous to DGGE, termed temperature gradient gel electrophoresis (TGGE), uses a thermal gradient rather than a chemical denaturant gradient (Scholz, et al., Hum. Mol. Genet. 2:2155, 1993). TGGE requires the use of specialized equipment which can generate a temperature gradient perpendicularly oriented relative to the electrical field. TGGE can detect mutations in relatively small fragments of DNA therefore scanning of large gene segments requires the use of multiple PCR products prior to running the gel.

Single-Strand Conformation Polymorphism (SSCP): Another common method, called “Single-Strand Conformation Polymorphism” (SSCP) was developed by Hayashi, Sekya and colleagues (reviewed by Hayashi, PCR Meth. Appl., 1:34-38, 1991) and is based on the observation that single strands of nucleic acid can take on characteristic conformations in non-denaturing conditions, and these conformations influence electrophoretic mobility. The complementary strands assume sufficiently different structures that one strand may be resolved from the other. Changes in sequences within the fragment will also change the conformation, consequently altering the mobility and allowing this to be used as an assay for sequence variations (Orita, et al., Genomics 5:874-879, 1989).

The SSCP process involves denaturing a DNA segment (e.g., a PCR product) that is labeled on both strands, followed by slow electrophoretic separation on a non-denaturing polyacrylamide gel, so that intra-molecular interactions can form and not be disturbed during the run. This technique is extremely sensitive to variations in gel composition and temperature. A serious limitation of this method is the relative difficulty encountered in comparing data generated in different laboratories, under apparently similar conditions.

Dideoxy fingerprinting (ddF): The dideoxy fingerprinting (ddF) is another technique developed to scan genes for the presence of mutations (Liu and Sommer, PCR Methods Appli., 4:97, 1994). The ddF technique combines components of Sanger dideoxy sequencing with SSCP. A dideoxy sequencing reaction is performed using one dideoxy terminator and then the reaction products are electrophoresed on nondenaturing polyacrylamide gels to detect alterations in mobility of the termination segments as in SSCP analysis. While ddF is an improvement over SSCP in terms of increased sensitivity, ddF requires the use of expensive dideoxynucleotides and this technique is still limited to the analysis of fragments of the size suitable for SSCP (i.e., fragments of 200-300 bases for optimal detection of mutations).

Pyrosequencing™ analysis (Pyrosequencing, Inc. Westborough, Mass., USA): This technique is based on the hybridization of a sequencing primer to a single stranded, PCR-amplified, DNA template in the presence of DNA polymerase, ATP sulfurylase, luciferase and apyrase enzymes and the adenosine 5′ phosphosulfate (APS) and luciferin substrates. In the second step the first of four deoxynucleotide triphosphates (dNTP) is added to the reaction and the DNA polymerase catalyzes the incorporation of the deoxynucleotide triphosphate into the DNA strand, if it is complementary to the base in the template strand. Each incorporation event is accompanied by release of pyrophosphate (PPi) in a quantity equimolar to the amount of incorporated nucleotide. In the last step the ATP sulfurylase quantitatively converts PPi to ATP in the presence of adenosine 5′ phosphosulfate. This ATP drives the luciferase-mediated conversion of luciferin to oxyluciferin that generates visible light in amounts that are proportional to the amount of ATP. The light produced in the luciferase-catalyzed reaction is detected by a charge coupled device (CCD) camera and seen as a peak in a Pyrogram™. Each light signal is proportional to the number of nucleotides incorporated.

Acycloprime™ analysis (Perkin Elmer, Boston, Mass., USA): This technique is based on fluorescent polarization (FP) detection. Following PCR amplification of the sequence containing the SNP of interest, excess primer and dNTPs are removed through incubation with shrimp alkaline phosphatase (SAP) and exonuclease I. Once the enzymes are heat inactivated, the Acycloprime-FP process uses a thermostable polymerase to add one of two fluorescent terminators to a primer that ends immediately upstream of the SNP site. The terminator(s) added are identified by their increased FP and represent the allele(s) present in the original DNA sample. The Acycloprime process uses AcycloPol™, a novel mutant thermostable polymerase from the Archeon family, and a pair of AcycloTerminators™ labeled with R110 and TAMRA, representing the possible alleles for the SNP of interest. AcycloTerminator™ non-nucleotide analogs are biologically active with a variety of DNA polymerases. Similarly to 2′,3′-dideoxynucleotide-5′-triphosphates, the acyclic analogs function as chain terminators. The analog is incorporated by the DNA polymerase in a base-specific manner onto the 3′-end of the DNA chain, and since there is no 3′-hydroxyl, is unable to function in further chain elongation. It has been found that AcycloPol has a higher affinity and specificity for derivatized AcycloTerminators than various Taq mutant have for derivatized 2′,3′-dideoxynucleotide terminators.

Reverse dot blot: This technique uses labeled sequence specific oligonucleotide probes and unlabeled nucleic acid samples. Activated primary amine-conjugated oligonucleotides are covalently attached to carboxylated nylon membranes. After hybridization and washing, the labeled probe, or a labeled fragment of the probe, can be released using oligomer restriction, i.e., the digestion of the duplex hybrid with a restriction enzyme. Circular spots or lines are visualized colorimetrically after hybridization through the use of streptavidin horseradish peroxidase incubation followed by development using tetramethylbenzidine and hydrogen peroxide, or via chemiluminescence after incubation with avidin alkaline phosphatase conjugate and a luminous substrate susceptible to enzyme activation, such as CSPD, followed by exposure to x-ray film.

LightCycler™ Analysis (Roche, Indianapolis, Ind., USA)—The LightCycler™ instrument consists of a thermocycler and a fluorimeter component for on-line detection. PCR-products formed by amplification are detected on-line through fluorophores coupled to two sequence-specific oligonucleotide hybridization probes. One of the oligonucleotides has a fluorescein label at its 3′-end (donor oligonucleotide) and the other oligonucleotide is labeled with LightCycler™-Red 640 at its 5′-end (acceptor oligonucleotide). When both labeled DNA-probes are hybridized to their template, energy is transferred from the donor fluorophore to the acceptor fluorophore following the excitation of the donor fluorophore using an external light source with a specific wavelength. The light that is emitted by the acceptor fluorophore can be detected at a defined wavelength. The intensity of this light signal is proportional to the amount of PCR-product.

It will be appreciated that advances in the field of SNP detection have provided additional accurate, easy, and inexpensive large-scale SNP genotyping techniques, such as dynamic allele-specific hybridization (DASH, Howell, W. M. et al., 1999. Dynamic allele-specific hybridization (DASH). Nat. Biotechnol. 17: 87-8), microplate array diagonal gel electrophoresis [MADGE, Day, I. N. et al., 1995. High-throughput genotyping using horizontal polyacrylamide gels with wells arranged for microplate array diagonal gel electrophoresis (MADGE). Biotechniques. 19: 830-5], the TaqMan system (Holland, P. M. et al., 1991. Detection of specific polymerase chain reaction product by utilizing the 5′→3′ exonuclease activity of Thermus aquaticus DNA polymerase. Proc Natl Acad Sci USA. 88: 7276-80), as well as various DNA “chip” technologies such as the GeneChip microarrays (e.g., Affymetrix SNP chips) which are disclosed in U.S. Pat. No. 6,300,063 to Lipshutz, et al. 2001, which is fully incorporated herein by reference, Genetic Bit Analysis (GBA™) which is described by Goelet, P. et al. (PCT Appl. No. 92/15712), peptide nucleic acid (PNA, Ren B, et al., 2004. Nucleic Acids Res. 32: e42) and locked nucleic acids (LNA, Latorra D, et al., 2003. Hum. Mutat. 22: 79-85) probes, Molecular Beacons (Abravaya K, et al., 2003. Clin Chem Lab Med. 41: 468-74), intercalating dye [Germer, S, and Higuchi, R. Single-tube genotyping without oligonucleotide probes. Genome Res. 9:72-78 (1999)], FRET primers (Solinas A et al., 2001. Nucleic Acids Res. 29: E96), AlphaScreen (Beaudet L, et al., Genome Res. 2001, 11(4): 600-8), SNPstream (Bell P A, et al., 2002. Biotechniques. Suppl.: 70-2, 74, 76-7), Multiplex minisequencing (Curcio M, et al., 2002. Electrophoresis. 23: 1467-72), SnaPshot (Turner D, et al., 2002. Hum Immunol. 63: 508-13), MassEXTEND (Cashman J R, et al., 2001. Drug Metab Dispos. 29: 1629-37), GOOD assay (Sauer S, and Gut I G. 2003. Rapid Commun. Mass. Spectrom. 17: 1265-72), Microarray minisequencing (Liljedahl U, et al., 2003. Pharmacogenetics. 13: 7-17), arrayed primer extension (APEX) (Tonisson N, et al., 2000. Clin. Chem. Lab. Med. 38: 165-70), Microarray primer extension (O'Meara D, et al., 2002. Nucleic Acids Res. 30: e75), Tag arrays (Fan J B, et al., 2000. Genome Res. 10: 853-60), Template-directed incorporation (TDI) (Akula N, et al., 2002. Biotechniques. 32: 1072-8), fluorescence polarization (Hsu T M, et al., 2001. Biotechniques. 31: 560, 562, 564-8), Colorimetric oligonucleotide ligation assay (OLA, Nickerson D A, et al., 1990. Proc. Natl. Acad. Sci. USA. 87: 8923-7), Sequence-coded OLA (Gasparini P, et al., 1999. J. Med. Screen. 6: 67-9), Microarray ligation, Ligase chain reaction, Padlock probes, Rolling circle amplification, Invader assay (reviewed in Shi M M. 2001. Enabling large-scale pharmacogenetic studies by high-throughput mutation detection and genotyping technologies. Clin Chem. 47: 164-72), coded microspheres (Rao K V et al., 2003. Nucleic Acids Res. 31: e66) and MassArray (Leushner J, Chiu N H, 2000. Mol. Diagn. 5: 341-80).

The 192R/Q polymorphs of the PON1 polypeptide can be detected using any biochemical or immunological methods known in the art.

An exemplary method for PON1 192R/Q polymorph detection is described in Example 3 herein below. This method is based on the knowledge that PON1 R192 undergoes higher enzymatic stimulation by NaCl [Eckerson, H. W., Romson, J., Wyte, C., and La Du, B. N. (1983) The human serum paraoxonase polymorphism: identification of phenotypes by their response to salts, Am J Hum Genet 35, 214-227]. These differences in isozymic properties allow serum samples to be phenotyped by dividing the paraoxonase activity in the presence of 1 M NaCl by the arylesterase activity.

Alternatively or additionally the 192R/Q polymorphs of the PON1 polypeptide can also be detected by an immunological detection method employed on a protein sample of the individual using an antibody or a fragment thereof which is capable of differentially binding (e.g., by antibody—antigen binding interaction) the polymorph of the present invention (192R/Q). As used herein the phrase “capable of differentially binding” refers to an antibody, which under the experimental conditions employed (as further described hereinunder) is capable of binding to only one polymorph (e.g., PON1 192Q) of the protein but not the other polymorph (e.g., PON1 192R) or vise versa. Antibodies useful in context of this embodiment of the invention can be prepared using methods of antibody preparation well known to one of ordinary skills in the art, using, for example, synthetic peptides derived from the various domains of the PON1 protein for vaccination of antibody producing animals and subsequent isolation of antibodies therefrom. Monoclonal antibodies specific to each of the PON1 protein polymorphs can also be prepared as is described, for example, in “Current Protocols in Immunology” Volumes I-III Coligan J. E., Ed. (1994); Stites et al. (Eds), “Basic and Clinical Immunology” (8th Edition), Appleton & Lange, Norwalk, Conn. (1994); Mishell and Shiigi (Eds), “Selected Methods in Cellular Immunology”, W.H. Freeman and Co., New York (1980).

The term “antibody” as used in the present invention includes intact molecules as well as functional fragments thereof, such as Fab, F(ab′)₂, and Fv that are capable of binding to macrophages. These functional antibody fragments are defined as follows: Fab, the fragment which contains a monovalent antigen-binding fragment of an antibody molecule, can be produced by digestion of whole antibody with the enzyme papain to yield an intact light chain and a portion of one heavy chain; Fab′, the fragment of an antibody molecule that can be obtained by treating whole antibody with pepsin, followed by reduction, to yield an intact light chain and a portion of the heavy chain; two Fab′ fragments are obtained per antibody molecule; (Fab′)₂, the fragment of the antibody that can be obtained by treating whole antibody with the enzyme pepsin without subsequent reduction; F(ab′)₂ is a dimer of two Fab′ fragments held together by two disulfide bonds; Fv, defined as a genetically engineered fragment containing the variable region of the light chain and the variable region of the heavy chain expressed as two chains; and single chain antibody (“SCA”), a genetically engineered molecule containing the variable region of the light chain and the variable region of the heavy chain, linked by a suitable polypeptide linker as a genetically fused single chain molecule.

Methods of making these fragments are known in the art. See for example, Harlow and Lane, Antibodies: A Laboratory Manual, Cold Spring Harbor Laboratory, New York, 1988, incorporated herein by reference.

As mentioned, the PON1 192Q polymorph can be detected in a protein sample of the individual using an immunological detection method. Such methods are fully explained in, for example, “Using Antibodies: A Laboratory Manual” [Ed Harlow, David Lane eds., Cold Spring Harbor Laboratory Press (1999)] and those familiar with the art will be capable of implementing the various techniques summarized hereinbelow as part of the present invention. All of the immunological techniques require antibodies specific to at least one of the PON1 192R/Q polymorphs. Immunological detection methods suited for use as part of the present invention include, but are not limited to, radio-immunoassay (RIA), enzyme linked immunosorbent assay (ELISA), western blot, immunohistochemical analysis, and fluorescence activated cell sorting (FACS).

As mentioned herein above, another test which may be performed either alone or in conjunction with the stability testing of the present invention in order to diagnose a subject with a lipid-related disorder is assaying a lactonase activity of serum PON1.

As used herein, the phrase “lactonase activity” refers to lactone hydrolysis activity, which typically, in accordance with this aspect of the present invention, refers to the hydrolysis of an ester bond of a lactone. Typically, an individual with a PON1 comprising a high lactonase activity is less susceptible to a lipid-related disorder.

Methods of determining a lactonase activity of an enzyme are well known in the art. These methods are typically effected by known biochemical assays such, for example, chromatographic assays (e.g., HPLC, TLC, GC, CPE) pH indicator assays, coupled assays (i.e., in these assays enzymes other than the one assayed are added to yield a measurable product; For example, the carboxylic acid product could be turned over by a dehydrogenase, and the change in concentration of NAD/NADH, or NADP/NADPH, monitored by absorbance or fluorescence), therm-ocalorimetric (i.e., monitoring changes in heat capacity), electrochemical assays (i.e., monitoring changes in redox potential) and/or spectrophotometric assays.

A typical enzyme assay is based on a chemical reaction which the tested enzyme catalyzes specifically. The chemical reaction is typically the conversion of a substrate or an analogue thereof into a product. The ability to detect minute changes in the levels, i.e., the concentration of either the substrate or the product enables the determination of the enzyme's activity both qualitatively and quantitatively, and even quantitatively determines the specificity of a particular substrate to the tested enzyme. In order to measure minute changes in the levels of the substrate and/or the product, these compounds should have a chemical and/or physical property which can be detected chemically or physically, such as a change in pH, molecular weight, color or another directly or indirectly measurable chemical and/or physical property.

Following is a description of exemplary lactonase assays which can be used in accordance with this aspect of the present invention.

pH indicator assays—Enzymatic assays which are based on pH indicators are typically used for measuring lactonase activity with aliphatic lactones. This may be achieved using the continuous pH-sensitive calorimetric assay (i.e., measuring the intensity of color generated by a pH indicator) such as described in Billecke et al. (2000) Drug Metab. Dispos. 28:1335-1342, using a SPECTRAmax® PLUS microplate reader (Molecular Devices, Sunnyvale, Calif.). The reactions (200 μl final volume) containing 2 mM HEPES, pH 8.0, 1 mM CaCl₂, 0.004% (w/v) Phenol Red, and diluted/non-diluted PON containing sample (e.g., serum sample, diluted 100-1000 fold) are initiated with 2 μl of 100 mM substrate solution in methanol and are carried out at 37° C. for 3-10 minutes. The rates are calculated from the slopes of the absorbance decrease at 558 nm with correction at 475 nm (isosbestic point) using a rate factor (mOD/μmol H⁺) estimated from a standard curve generated with known amounts of HCL. The spontaneous hydrolysis of the lactones and acidification by atmospheric CO₂ are preferably corrected for by carrying out parallel reactions with the same volume of storage buffer instead of enzyme.

Alternatively, proton release resulting from carboxylic acid formation can be monitored using the pH indicator cresol purple. The reactions are performed at pH 8.0-8.3 in bicine buffer 2.5 mM, containing 1 mM CaCl₂ and 0.2 M NaCl. The reaction mixture contains 0.2-0.3 mM cresol red (from a 60 mM stock in DMSO). Upon mixture of the substrate with the enzyme sample, the decrease in absorbance at 577 nm is monitored in a microtiter plate reader. The assay requires in situ calibration with acetic acid (standard acid titration curve), which gives the rate factor (−OD/mole of H⁺).

HPLC analysis—Hydrolysis of various lactone substrates can be detected by HPLC analysis. Thus for example, the hydrolysis of acylhomoserine lactones (AHLs) can be analyzed by HPLC (e.g., Waters 2695 system equipped with Waters 2996 photodiode array detector set at 197 nm using Supelco Discovery C-18 column (250×4.6 mm, 5 μm particles). Enzymatic reactions are carried at room temperature in 50 μl volume of 25 mM Tris-HCl, pH 7.4, 1 mM CaCl₂, 25 μM AHL (e.g., from 2 mM stock solution in methanol) and diluted/non-diluted PON containing sample (e.g., serum sample, diluted 100-1000 fold). Reactions are stopped with 50 μl acetonitrile (ACN) and centrifuged to remove the protein. Supernatants (40 μl) are loaded onto an HPLC system and eluted isocratically with 85% CAN/0.2% acetic acid (tetradeca-homoserine lactone). 0.75% CAN/0.2% acetic acid (dodeca-homoserine lactone), 50% CAN/).2% acetic acid (hepta-homoserine lactone), or 20% CAN/0.2% acetic acid (3-oxo-hexanoyl homoserine lactone).

The hydrolysis of the statin lactones (mevastatin, lovastatin and simvastatin) can be analyzed by high performance liquid chromatography (HPLC) such as by using a Beckman System Gold HPLC with a Model 126 Programmable Solvent Module, a Model 168 Diode Array Detector set at 238 nm, a Model 7125 Rheodyne manual injector valve with a 20 μl loop, and a Beckman ODS Ultrasphere column (C 18, 250×4.6 mm, 5 μm). Lovastatin (Mevacor) and simvastatin can be purchased as 20 mg tablets from Merck, from which the lactones are extracted with chloroform, evaporated to dryness and redissolved in methanol. Mevastatin can be purchased from Sigma.

In a final volume of 1 ml, 10-200 μl of enzyme solution and 10 μl of substrate solution in methanol (0.5 mg/ml) are incubated at 25° C. in 50 mM Tris/HCl (pH 7.6), 1 mM CaCl₂. Aliquots (100 μl) are removed at specified times and added to acetonitrile (100 μl), vortexed, and centrifuged for one minute at maximum speed (Beckman microfuge). The supernatants are poured into new tubes, capped and stored on ice until HPLC analysis.

Samples are eluted isocratically at a flow rate of 1.0 ml/min with a mobile phase consisting of the following: A=acetic acid/acetonitrile/water (2:249:249, v/v/v) and B=acetonitrile, in A/B ratios of 50/50, 45/55 and 40/60 for mevastatin, lovastatin and simvastatin, respectively.

Spectrophotometric assays—In these assays the consumption of the substrate and/or the formation of the product can be measured by following changes in the concentrations of a spectrophotometrically detectable moiety that is formed during the enzymatic catalysis. Examples of spectrophotometric assays include, without limitation, phosphorescence assays, fluorescence assays, chromogenic assays, luminescence assays and illuminiscence assays.

Phosphorescence assays monitor changes in the luminescence produced by a spectrophotometrically detectable moiety after absorbing radiant energy or other types of energy. Phosphorescence is distinguished from fluorescence in that it continues even after the radiation causing it has ceased.

Fluorescence assays monitor changes in the luminescence produced by a spectrophotometrically detectable moiety under stimulation or excitation by light or other forms of electromagnetic radiation or by other means. The light is given off only while the stimulation continues; in this the phenomenon differs from phosphorescence, in which light continues to be emitted after the excitation by other radiation has ceased.

Chromogenic assays monitor changes in color of the assay medium produced by a spectrophotometrically detectable moiety which has a characteristic wavelength.

Luminescence assays monitor changes in the luminescence produced a chemiluminescent and therefore spectrophotometrically detectable moiety generated or consumed during the enzymatic reaction. Luminescence is caused by the movement of electrons within a substance from more energetic states to less energetic states.

According to a preferred embodiment of the present invention, determining a lactonase activity of a PON enzyme is effected by a spectrophotometric assay. Such an assay, according to further preferred embodiments of the present invention, utilizes substrates that comprise one or more lactones and which are capable of forming one or more spectrophotometrically detectable moieties. The enzyme is contacted with such substrates and the amount of the detectable moiety is measured. A particularly preferred substrate, according to the embodiment of this aspect of the present invention is TBBL.

According to one embodiment, the lactonase activity is normalized to the total enzyme levels.

As used herein the phrase “normalized lactonase activity” refers to a lactonase activity of PON1 as a function of total PON1.

Both determination of lactonase activity and total PON1 levels have been described hereinabove.

An exemplary method of determining a normalized lactonase activity is determining the ratio of the lipoprotein-stimulated lactonase hydrolysis (e.g. TBBL) to dihydrocoumarin hydrolysis. According to this aspect of the present invention, a normalized lactonase activity below a predetermined threshold is indicative of a lipid-related disorder.

Determination of accurate thresholds may be effected by measuring the normalized lactonase activity in a statistically relevant group of individuals with lipid related disorders and comparing the activities with those measured in statistically relevant groups of healthy individuals.

It will be appreciated that the agents utilized by the methods described hereinabove of diagnosing a lipid related disorder can form a part of a kit.

Such a kit includes at least one agent for determining a stability of a serum PON1:lipoprotein complex, such as a PON1 inactivator e.g. NTA, β-mercaptoethanol or both. The kit may also comprise agents for determining a total amount of PON1: lipoprotein complex. In addition, the kit may also comprise agents for determining a presence or absence in a homozygous or heterozygous form, of the PON1 575A allele and/or the PON1 Q192 polymorph and/or for analyzing the lactonase activity of PON1. Examples of such agents include HDL stimulated (e.g. TBBL) and HDL non-stimulated lactones (e.g. dihydrocoumarin).

According to preferred embodiments the kits further includes packaging material and a notification in or on the packaging material identifying the kits for use in determining if an individual is predisposed to a lipid-related disorder.

The kit also includes the appropriate instructions for use and labels indicating FDA approval for use in diagnostics.

Using an in-vitro model, the present inventors have shown that the PON1 192R polymorph is associated with an increased antiatherogenic potency as compared to the PON1 192Q polymorph as measured by an increase in cholesterol efflux from macrophages (FIG. 12).

Thus according to another aspect of the present invention, there is provided a method of treating a lipid-related disorder, comprising administering to a subject in need thereof a therapeutically effective amount an agent for increasing expression of an arginine-containing polymorph at position 192 of a PON1 polypeptide as set forth in SEQ ID NO: 14.

Herein, the term “treating” includes abrogating, substantially inhibiting, slowing or reversing the progression of a lipid related disorder or substantially preventing the onset of a lipid related disorder or symptoms of a lipid related disorder Preferably, treating cures, e.g., substantially eliminates, the symptoms associated with the lipid related disorder.

As mentioned herein above the lipid related disorders of the present invention are associated with an altered PON1 activity. Examples of such disorders include, but are not limited to cardiovascular disorders [Costa et al. (2005); Mackness et al. (2004) The role of paraoxonase 1 activity in cardiovascular disease: potential for therapeutic intervention. Am J Cardiovasc Drugs. 2004; 4(4):211-7; Durrington et al (2001) Paraoxonase and atherosclerosis. Arterioscler Thromb Vasc Biol. 2001 21(4):473-80]; pancreatic disorders and neurological disorders. Examples of cardiovascular disorders include but are not limited to atherosclerosis, coronary heart disease, myocardial infarction, peripheral vascular diseases, venous thromboembolism, stroke and pulmonary embolism.

Other examples of lipid related disorders that are associated with an altered PON1 activity include insulin-dependent (type I) and non-insulin-dependent (type II) diabetes and Alzheimer's disease (Dantoine et al. 2002 Paraoxonase 1 activity: a new vascular marker of dementia? Ann N Y Acad. Sci. 2002 November; 977:96-101). Decreased PON activity has also been found in patients with chronic renal failure, rheumatoid arthritis or Fish-Eye disease (characterized by severe corneal opacities). Hyperthyroidism is also associated with lower serum PON activity, liver diseases, Alzheimer's disease, and vascular dementia. Lower PON activity is also observed in infectious diseases (e.g., during acute phase response). Abnormally low PON levels are also associated with exposure to various exogenous compounds such as environmental chemicals (e.g., metals such as, cobalt, cadmium, nickel, zinc, copper, barium, lanthanum, mercurials; dichloroacetic acid, carbon tetrachloride), drugs (e.g., cholinergic muscarinic antagonist, pravastatin, simvastatin, fluvastatin, alcohol). As mentioned reduced PON levels is also a characteristic of various physiological conditions such as pregnancy, and old age and may be indicative of a subject general health states. For example, smokers exhibit low serum PON1 activity and physical exercise is known to restore PON1 levels in smokers.

An exemplary agent that increases expression of an arginine-containing polymorph at position 192 of PON1 is a polypeptide as set forth in SEQ ID NO: 14.

The term “polypeptide” as used herein encompasses native polypeptides (either degradation products, synthetically synthesized polypeptides or recombinant polypeptides) and peptidomimetics (typically, synthetically synthesized polypeptides), as well as peptoids and semipeptoids which are polypeptide analogs, which may have, for example, modifications rendering the polypeptides more stable while in a body or more capable of penetrating into cells. Such modifications include, but are not limited to N terminus modification, C terminus modification, polypeptide bond modification, including, but not limited to, CH2-NH, CH2-S, CH2-S═O, O═C—NH, CH2-O, CH2-CH2, S═C—NH, CH═CH or CF═CH, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

Polypeptide bonds (—CO—NH—) within the polypeptide may be substituted, for example, by N-methylated bonds (—N(CH3)-CO—), ester bonds (—C(R)H—C—O—O—C(R)—N—), ketomethylen bonds (—CO—CH2-), α-aza bonds (—NH—N(R)—CO—), wherein R is any alkyl, e.g., methyl, carba bonds (—CH2-NH—), hydroxyethylene bonds (—CH(OH)—CH2-), thioamide bonds (—CS—NH—), olefinic double bonds (—CH═CH—), retro amide bonds (—NH—CO—), polypeptide derivatives (—N(R)—CH2-CO—), wherein R is the “normal” side chain, naturally presented on the carbon atom.

These modifications can occur at any of the bonds along the polypeptide chain and even at several (2-3) at the same time.

Natural aromatic amino acids, Trp, Tyr and Phe, may be substituted for synthetic non-natural acid such as Phenylglycine, TIC, naphthylelanine (Nol), ring-methylated derivatives of Phe, halogenated derivatives of Phe or o-methyl-Tyr.

In addition to the above, the polypeptides of the present invention may also include one or more modified amino acids or one or more non-amino acid monomers (e.g. fatty acids, complex carbohydrates etc).

As used herein in the specification and in the claims section below the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

Since the present polypeptides are preferably utilized in therapeutics which require the polypeptides to be in a soluble form, the polypeptides of the present invention may include one or more non-natural or natural polar amino acids, including but not limited to serine and threonine which are capable of increasing polypeptide solubility due to their hydroxyl-containing side chain.

The polypeptides of the present invention are preferably utilized in a linear form, although it will be appreciated that in cases where cyclicization does not severely interfere with polypeptide characteristics, cyclic forms of the polypeptide can also be utilized.

The polypeptides of present invention can be biochemically synthesized such as by using standard solid phase techniques. These methods include exclusive solid phase synthesis, partial solid phase synthesis methods, fragment condensation, classical solution synthesis. These methods are preferably used when the polypeptide is relatively short (i.e., 10 kDa) and/or when it cannot be produced by recombinant techniques (i.e., not encoded by a nucleic acid sequence) and therefore involves different chemistry.

Solid phase polypeptide synthesis procedures are well known in the art and further described by John Morrow Stewart and Janis Dillaha Young, Solid Phase Polypeptide Syntheses (2nd Ed., Pierce Chemical Company, 1984).

Synthetic polypeptides can be purified by preparative high performance liquid chromatography [Creighton T. (1983) Proteins, structures and molecular principles. WH Freeman and Co. N.Y.] and the composition of which can be confirmed via amino acid sequencing.

Recombinant techniques are preferably used to generate the polypeptides of the present invention since these techniques are better suited for generation of relatively long polypeptides (e.g., longer than 20 amino acids) and large amounts thereof. Such recombinant techniques are described by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680 and Brogli et al., (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463.

To produce a polypeptide of the present invention using recombinant technology, a polynucleotide encoding a polypeptide of the present invention is ligated into a nucleic acid expression vector, which comprises the polynucleotide sequence under the transcriptional control of a cis-regulatory sequence (e.g., promoter sequence) suitable for directing constitutive, tissue specific or inducible transcription of the polypeptides of the present invention in the host cells.

The polynucleotide sequence may be provided in the form of an RNA sequence, a complementary polynucleotide sequence (cDNA), a genomic polynucleotide sequence and/or a composite polynucleotide sequences (e.g., a combination of the above).

As used herein the phrase “complementary polynucleotide sequence” refers to a sequence, which results from reverse transcription of messenger RNA using a reverse transcriptase or any other RNA dependent DNA polymerase. Such a sequence can be subsequently amplified in vivo or in vitro using a DNA dependent DNA polymerase.

As used herein the phrase “genomic polynucleotide sequence” refers to a sequence derived (isolated) from a chromosome and thus it represents a contiguous portion of a chromosome.

As used herein the phrase “composite polynucleotide sequence” refers to a sequence, which is at least partially complementary and at least partially genomic. A composite sequence can include some exonal sequences required to encode the polypeptide of the present invention, as well as some intronic sequences interposing therebetween. The intronic sequences can be of any source, including of other genes, and typically will include conserved splicing signal sequences. Such intronic sequences may further include cis acting expression regulatory elements.

According to an embodiment of this aspect of the present invention the polynucleotide is set forth in SEQ ID NO: 13.

Polynucleotides of the present invention may be prepared using any method or procedure known in the art for ligation of two different DNA sequences. See, for example, “Current Protocols in Molecular Biology”, eds. Ausubel et al., John Wiley & Sons, 1992.

As mentioned hereinabove, polynucleotide sequences of the present invention are inserted into expression vectors (i.e., a nucleic acid construct) to enable expression of the recombinant polypeptide. The expression vector of the present invention includes additional sequences which render this vector suitable for replication and integration in prokaryotes, eukaryotes, or preferably both (e.g., shuttle vectors). Typical cloning vectors contain transcription and translation initiation sequences (e.g., promoters, enhances) and transcription and translation terminators (e.g., polyadenylation signals).

A variety of prokaryotic or eukaryotic cells can be used as host-expression systems to express the polypeptides of the present invention. These include, but are not limited to, microorganisms, such as bacteria transformed with a recombinant bacteriophage DNA, plasmid DNA or cosmid DNA expression vector containing the polypeptide coding sequence; yeast transformed with recombinant yeast expression vectors containing the polypeptide coding sequence; plant cell systems infected with recombinant virus expression vectors (e.g., cauliflower mosaic virus, CaMV; tobacco mosaic virus, TMV) or transformed with recombinant plasmid expression vectors, such as Ti plasmid, containing the polypeptide coding sequence.

In bacterial systems, a number of expression vectors can be advantageously selected depending upon the use intended for the polypeptide expressed. For example, when large quantities of polypeptide are desired, vectors that direct the expression of high levels of the protein product, possibly as a fusion with a hydrophobic signal sequence, which directs the expressed product into the periplasm of the bacteria or the culture medium where the protein product is readily purified may be desired. Certain fusion protein engineered with a specific cleavage site to aid in recovery of the polypeptide may also be desirable. Such vectors adaptable to such manipulation include, but are not limited to, the pET series of E. coli expression vectors [Studier et al., Methods in Enzymol. 185:60-89 (1990)].

In yeast, a number of vectors containing constitutive or inducible promoters can be used, as disclosed in U.S. Pat. No. 5,932,447. Alternatively, vectors can be used which promote integration of foreign DNA sequences into the yeast chromosome.

In cases where plant expression vectors are used, the expression of the polypeptide coding sequence can be driven by a number of promoters. For example, viral promoters such as the 35S RNA and 19S RNA promoters of CaMV [Brisson et al., Nature 310:511-514 (1984)], or the coat protein promoter to TMV [Takamatsu et al., EMBO J. 6:307-311 (1987)] can be used. Alternatively, plant promoters can be used such as, for example, the small subunit of RUBISCO [Coruzzi et al., EMBO J. 3:1671-1680 (1984); and Brogli et al., Science 224:838-843 (1984)] or heat shock promoters, e.g., soybean hsp17.5-E or hsp17.3-B [Gurley et al., Mol. Cell. Biol. 6:559-565 (1986)]. These constructs can be introduced into plant cells using Ti plasmid, Ri plasmid, plant viral vectors, direct DNA transformation, microinjection, electroporation and other techniques well known to the skilled artisan. See, for example, Weissbach & Weissbach [Methods for Plant Molecular Biology, Academic Press, NY, Section VIII, pp 421-463 (1988)]. Other expression systems such as insects and mammalian host cell systems, which are well known in the art, can also be used by the present invention.

It will be appreciated that other than containing the necessary elements for the transcription and translation of the inserted coding sequence (encoding the polypeptide), the expression construct of the present invention can also include sequences engineered to optimize stability, production, purification, yield or activity of the expressed polypeptide.

Various methods can be used to introduce the expression vector of the present invention into the host cell system. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et al. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

Transformed cells are cultured under effective conditions, which allow for the expression of high amounts of recombinant polypeptide. Effective culture conditions include, but are not limited to, effective media, bioreactor, temperature, pH and oxygen conditions that permit protein production. An effective medium refers to any medium in which a cell is cultured to produce the recombinant polypeptide of the present invention. Such a medium typically includes an aqueous solution having assimilable carbon, nitrogen and phosphate sources, and appropriate salts, minerals, metals and other nutrients, such as vitamins. Cells of the present invention can be cultured in conventional fermentation bioreactors, shake flasks, test tubes, microtiter dishes and petri plates. Culturing can be carried out at a temperature, pH and oxygen content appropriate for a recombinant cell. Such culturing conditions are within the expertise of one of ordinary skill in the art.

Depending on the vector and host system used for production, resultant polypeptides of the present invention may either remain within the recombinant cell, secreted into the fermentation medium, secreted into a space between two cellular membranes, such as the periplasmic space in E. coli; or retained on the outer surface of a cell or viral membrane.

Following a predetermined time in culture, recovery of the recombinant polypeptide is effected.

The phrase “recovering the recombinant polypeptide” used herein refers to collecting the whole fermentation medium containing the polypeptide and need not imply additional steps of separation or purification.

Thus, polypeptides of the present invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

To facilitate recovery, the expressed coding sequence can be engineered to encode the polypeptide of the present invention and fused cleavable moiety. Such a fusion protein can be designed so that the polypeptide can be readily isolated by affinity chromatography; e.g., by immobilization on a column specific for the cleavable moiety. Where a cleavage site is engineered between the polypeptide and the cleavable moiety, the polypeptide can be released from the chromatographic column by treatment with an appropriate enzyme or agent that specifically cleaves the fusion protein at this site [e.g., see Booth et al., Immunol. Lett. 19:65-70 (1988); and Gardella et al., J. Biol. Chem. 265:15854-15859 (1990)].

The polypeptide of the present invention is preferably retrieved in “substantially pure” form.

As used herein, the phrase “substantially pure” refers to a purity that allows for the effective use of the protein in the applications described herein.

In addition to being synthesizable in host cells, the polypeptide of the present invention can also be synthesized using in vitro expression systems. These methods are well known in the art and the components of the system are commercially available.

The polypeptides of the present invention can be provided to the individual per se, or as part of a pharmaceutical composition where it is mixed with a pharmaceutically acceptable carrier.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the polypeptide or antibody preparation, which is accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases. One of the ingredients included in the pharmaceutically acceptable carrier can be for example polyethylene glycol (PEG), a biocompatible polymer with a wide range of solubility in both organic and aqueous media (Mutter et al. (1979).

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, transnasal, intestinal or parenteral delivery, including intramuscular, subcutaneous and intramedullary injections as well as intrathecal, direct intraventricular, intravenous, intraperitoneal, intranasal, or intraocular injections.

Alternately, one may administer the preparation in a local rather than systemic manner, for example, via injection of the preparation directly into a specific region of a patient's body.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the invention may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The preparations described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The preparation of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients effective to prevent, alleviate or ameliorate symptoms of disease or prolong the survival of the subject being treated.

Determination of a therapeutically effective amount is well within the capability of those skilled in the art.

For any preparation used in the methods of the invention, the therapeutically effective amount or dose can be estimated initially from in vitro assays. For example, a dose can be formulated in animal models and such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. [See e.g., Fingl, et al., (1975) “The Pharmacological Basis of Therapeutics”, Ch. 1 p. 1].

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions including the preparation of the present invention formulated in a compatible pharmaceutical carrier may also be prepared, placed in an appropriate container, and labeled for treatment of an indicated condition.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

Alternatively, these peptides can be manufactured within the target cell by administering a nuclear acid construct of the peptide. It will be appreciated that the nucleic acid construct can be administered to the individual employing any suitable mode of administration, described hereinbelow (i.e. in vivo gene therapy). Alternatively, the nucleic acid construct can be introduced into a suitable cell using an appropriate gene delivery vehicle/method (transfection, transduction, etc.) and an appropriate expression system. The modified cells are subsequently expanded in culture and returned to the individual (i.e. ex vivo gene therapy). Examples of suitable constructs include, but are not limited to, pcDNA3, pcDNA3.1 (+/−), pGL3, PzeoSV2 (+/−), pDisplay, pEF/myc/cyto, pCMV/myc/cyto each of which is commercially available from Invitrogen Co. (www.invitrogen.com). Examples of retroviral vector and packaging systems are those sold by Clontech, San Diego, Calif., including Retro-X vectors pLNCX and pLXSN, which permit cloning into multiple cloning sites and transcription of the transgene is directed from the CMV promoter. Vectors derived from Mo-MuLV are also included such as pBabe, where the transgene will be transcribed from the 5′LTR promoter.

Currently preferred in vivo nucleic acid transfer techniques include infection with viral or transfection with a non-viral constructs. The former includes, but is not limited to the adenovirus, lentivirus, Herpes simplex I virus and adeno-associated virus (AAV) whilst the latter includes, but is not limited to lipid-based systems. Useful lipids for lipid-mediated transfer of the gene are, for example, DOTMA, DOPE, and DC-Chol [Tonkinson et al., Cancer Investigation, 14(1): 54-65 (1996)]. Recently, it has been shown that Chitosan can be used to deliver nucleic acids to the intestine cells (Chen J. (2004) World J Gastroenterol 10(1):112-116). The most preferred constructs for use in gene therapy are viruses, most preferably adenoviruses, AAV, lentiviruses, or retroviruses. A viral construct such as a retroviral construct includes at least one transcriptional promoter/enhancer or locus-defining element(s), or other elements that control gene expression by other means such as alternate splicing, nuclear RNA export, or post-transcriptional modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide from a host cell in which it is placed. Preferably, the signal sequence for this purpose is a mammalian signal sequence or the signal sequence of the peptide variants of the present invention. Optionally, the construct may also include a signal that directs polyadenylation, as well as one or more restriction site and a translation termination sequence. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

It will be appreciated that the agent that increases expression of an arginine-containing polymorph at position 192 of PON1 is an oligonucleotide which may be introduced to the subject using the well known “gene knock-in strategy” which will result in the formation of a PON1 192R.

Additional objects, advantages, and novel features of the present invention will become apparent to one ordinarily skilled in the art upon examination of the following examples, which are not intended to be limiting. Additionally, each of the various embodiments and aspects of the present invention as delineated herein above and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### HDL Binding Properties of Recombinant PON1, Recombinant PON1-192Q and Recombinant PON1-192R

The HDL binding properties of three PON1 isozymes were compared by examining their stability, binding affinity, and stimulation of their lactonase activity by rHDL-apoA-I. The three isozymes were recombinant PON1 (rePON1), where position 192 contains lysine, recombinant PON1-192Q, where position 192 contains glutamine and recombinant-192R, where position 192 contains arginine. rePON1 is a close homologue of rabbit PON1 (95% amino acid identity), which is highly similar to human PON1. Recombinant PON1-192Q and recombinant PON1-192R were generated to mimic the two naturally occurring human isozymes.

Materials and Methods

Preparation of recombinant PON1, recombinant PON1-192Q and recombinant PON1-192R: A recombinant, wild-type PON1 variant dubbed G3C9, fused to a His₈-tag at the carboxy terminus (dubbed, rePON1-192K; SEQ ID NO:2) was used for the in vitro system [Gaidukov, L. and D. S. Tawfik, Biochemistry, 2005. 44(35): p. 11843-54]. The 192R (SEQ ID NO:4) and 192Q (SEQ ID NO:6) isozymes were generated by PCR [1] Khersonsky, O. and D. S. Tawfik, J Biol Chem, 2006] and cloned into a modified pET32b vector (pET32-trx) as described [Aharoni, A., et al., Proc Natl Acad Sci USA, 2004. 101(2): p. 482-7]. The rePON1 variants were expressed in E. coli and purified as previously described [Gaidukov, L. and D. S. Tawfik, Biochemistry, 2005. 44(35): p. 11843-54].

Preparation of rHDL-apoA-I: Human apolipoprotein A-I (apoA-I) Gene in pET20b vector [Oda, M. N., et al., Biochemistry, 2001. 40(6): p. 1710-8] was kindly provided by Michael Oda (Oakland Research Institute). Rabbit apoA-I was cloned into pET20b vector as described [Gaidukov, L. and D. S. Tawfik, Biochemistry, 2005. 44(35): p. 11843-54]. Both ApoA-Is were expressed in E. coli and purified as described [Gaidukov, L. and D. S. Tawfik, Biochemistry, 2005. 44(35): p. 11843-54]. Discoidal rHDL containing egg PC, free cholesterol (FC) and apoA-I at a starting molar ratio of 100/5/1, were prepared by the cholate dialysis method as previously described [Gaidukov, L. and D. S. Tawfik, Biochemistry, 2005. 44(35): p. 11843-54] with the following variations. Purified apoA-Is were resuspended in 3 M guanidine hydrochloride solution, briefly dialyzed against TBS, and immediately added to the PC/FC mixture. Anion exchange resin (diethyl aminoethyl, DEAE, Whatman) was added to three last buffer exchanges to remove residual sodium deoxycholate.

A. Stability of the Full Length Variants of PON1 192 Isozymes

Inactivation rates of rePON1s were measured as described [Gaidukov, L. and D. S. Tawfik, Biochemistry, 2005. 44(35): p. 11843-54]. Briefly, rePON1 samples were delipidated using Bio-Beads SM-2 (BioRad) and incubated in buffer, or with 50-fold molar excess of rHDL-apoA-I. EDTA (10 mM) and β-mercaptoethanol (20 mM) were added, and samples were incubated at 37° C. Residual activity at different time points was determined with 2 mM phenyl acetate, and inactivation rates were fitted to either mono- or double-exponentials [Gaidukov, L. and D. S. Tawfik, Biochemistry, 2005. 44(35): p. 11843-54].

B. Affinity Measurements of the Truncated Variants of PON1 192 Isozymes

Production of rePON1s: Truncated (Δ20-rePON1) variants of rePON1 (SEQ ID NO:8) and its 192R (SEQ ID NO:10) and 192Q (SEQ ID NO:12) isozymes were prepared as described [Gaidukov, L. and D. S. Tawfik, Biochemistry, 2005. 44(35): p. 11843-54][2]. The rePON1 variants were expressed in E. coli and purified as previously described [Gaidukov, L. and D. S. Tawfik, Biochemistry, 2005. 44(35): p. 11843-54].

Surface plasmon resonance (SPR) measurements: rHDL-apoA-I particles containing 0.7% of N-biotinyl-dipalmitoylphosphatidylethanolamine (N-biotynyl-DPPE; Avanti Polar Lipids) were prepared and purified as described [Gaidukov, L. and D. S. Tawfik, Biochemistry, 2005. 44(35): p. 11843-54]. SPR was performed on BIAcore 3000 (Uppsala, Sweden) as described [Gaidukov, L. and D. S. Tawfik, Biochemistry, 2005. 44(35): p. 11843-54]. Briefly, the biotinylated rHDL particles were adsorbed on streptavidin (SA5) chip, and Δ20-rePON1 isozymes were injected over the immobilized and blank surfaces to obtain the net binding response. Binding rate constants were obtained by fitting of association and dissociation phases to single exponentials as described [Gaidukov, L. and D. S. Tawfik, Biochemistry, 2005. 44(35): p. 11843-54].

C. Stimulation of Enzymatic Activity of PON1 192 Isozymes

Delipidated rePONs at 0.2 μM were incubated with a range of rHDL concentrations (0.1-10 μM) for 3 hrs at 37° C. Enzymatic activity was determined in activity buffer (50 mM Tris pH 8.0, 1 mM CaCl₂) with substrates at 1 mM concentrations as described [Gaidukov, L. and D. S. Tawfik, Biochemistry, 2005. 44(35): p. 11843-54]. 5-(thiobutyl)-butyrolactone (TBBL) was applied at 0.25 mM, and product formation was monitored spectrophotometrically at 412 nm using 5,5′-dithio-bis-2-nitrobenzoic acid (DTNB) as described [Khersonsky, O. and D. S. Tawfik, Chembiochem, 2006. 7(1): p. 49-53].

Results

The resultant inactivation profiles of wt rePON1 (SEQ ID NO: 2), rePON1-192Q (SEQ ID NO: 6) and rePON1-192R (SEQ ID NO: 4), were fitted to exponential curves as illustrated in FIG. 1. The inactivation rate constants which were derived from the curves are set forth in Table 1 herein below.

TABLE 1
Kinetic and equilibrium constants for the inactivation of the rePON1 192
isozymes in buffer and on apoA-I rHDL

| | apoA-I rHDL | | | Activity buffer | | |
|---|---|---|---|---|---|---|
| | A₂ (%)* | k₁^{inactiv} (hr⁻¹)* | k₂^{inactiv} (hr⁻¹)* | A₂ (%) | k₁^{inactiv} (hr⁻¹) | k₂^{inactiv} (hr⁻¹) |
| wt rePON1- | 100 | — | 0.01 | 31 | 4.5 | 0.2 |
| 192K (SEQ | | | | | | |
| ID NO: 2) | | | | | | |
| rePON1- | 90 | 0.9 | 0.01 | 31 | 5.5 | 0.2 |
| 192R (SEQ | | | | | | |
| ID NO: 4) | | | | | | |
| rePON1- | 70 | 1.1 | 0.01 | 24 | 5.2 | 0.3 |
| 192Q | | | | | | |
| (SEQ ID | | | | | | |
| NO: 6) | | | | | | |

*Prefixes 1 and 2 designate the first (fast) and the second (slow) phases of the inactivation, respectively. Each value represents the mean of two independent experiments. Standard deviations were less then 10% of parameter values.

Inactivation of wt rePON1-192K bound to rHDL-apoA-I followed a mono-exponential kinetics with a rate constant (k^{inactiv}) of 0.01 hr⁻¹, corresponding to a full (100%) association of PON1 with HDL [Gaidukov, L., and Tawfik, D. S. (2005) Biochemistry 44, 11843-11854]. In contrast, inactivation of rePON1-192Q followed a double-exponential regime with the first (fast) phase (k₁^{inactiv}=1.1 hr⁻¹) corresponding to free protein, and the second (slow) phase corresponding to the HDL-bound fraction, which is 100 times more stable and exhibits similar inactivation rate as wt rePON1-192K (k₂^{inactiv}=0.01 hr⁻¹) (Table 1). The difference between the two isozymes is, therefore, in the partition between the unbound and HDL-bound forms (namely, the degree of HDL binding), while the rate of inactivation of HDL-bound form is similar for both isozymes. The fast inactivation phase of rePON1-192Q constitutes 30% of the total amplitude, showing that only 70% of rePON1-192Q is HDL-bound under these conditions. The effect of replacing K192 by R was milder, with the fast phase of 10%, indicating that 90% of the protein is bound to HDL. In buffer, inactivation kinetics of the three isozymes followed a double-exponential regime with very similar kinetic rates and amplitudes of the phases, indicating that there is no difference in the intrinsic stability of the proteins.

The above results were further supported by affinity measurements using surface plasmon resonance. It was previously shown that HDL affinity of the intact rePON1 is very high (sub-nanomolar) and could not be determined accurately using surface plasmon resonance [Gaidukov, L., and Tawfik, D. S. (2005)Biochemistry 44, 11843-11854]. In contrast, binding affinity of the truncated variant lacking the first 20 amino acids of the hydrophobic N-terminus (Δ20-rePON1) could be amply determined. Therefore, the truncated variants of PON1 192 isozymes were prepared and their HDL binding affinities measured. Typical binding sensorgrams between Δ20-rePON1 isozymes and the immobilized HDL particles are portrayed in FIGS. 2A-C, and the derived rate and affinity constants are summarized in Table 2 herein below.

TABLE 2
Kinetic and Affinity Constants for the Binding of Δ20-rePON1 192 isozymes to apoA-I rHDL

| | | | | A₂ (amplitude) |
|---|---|---|---|---|
| | kₒₙ (s⁻¹ M⁻¹) | k_{off} (s⁻¹) | K_{d} (M) | (%) |
| wt Δ20-rePON1-192K | (2.0 ± 0.3) × 10⁵ | (2.0 ± 0.2) × 10⁻² | (1.0 ± 0.2) × 10⁻⁷ | 100 |
| Δ20-rePON1-192R | (2.6 ± 0.3) × 10⁵ | (2.9 ± 0.2) × 10⁻² | (1.1 ± 0.2) × 10⁻⁷ | 90 |
| Δ20-rePON1-192Q | (1.4 ± 0.5) × 10⁵ | (4.2 ± 0.2) × 10⁻² | (3.0 ± 0.6) × 10⁻⁷ | 70 |

Association and dissociation phases were fitted to a single exponential to give k^{obs}. kₒₙ was derived from the linear fit of kₒₙ^{obs} vs concentration (kₒₙ^{obs} = [rePON1] kₒₙ + k_{off}). k_{off} was derived directly from k_{off}^{obs} that was independent of PON1 concentration. Each value represents the mean and SD of two independent experiments.

Wt Δ20-rePON1-192K exhibited a 3-fold fold higher HDL binding affinity than the Q isozyme, mainly due to the slower dissociation rate. Δ20-rePON1-192R, on the other hand, exhibited rate and affinity constants very similar to the wt. Thus, a remarkable correlation was observed between the affinity and the stability of PON1 on HDL. Wt rePON1-192K and its R homolog show a 3-fold higher HDL affinity than the Q isozyme, and a 3-fold higher fraction of HDL-bound protein as revealed from the stability measurements.

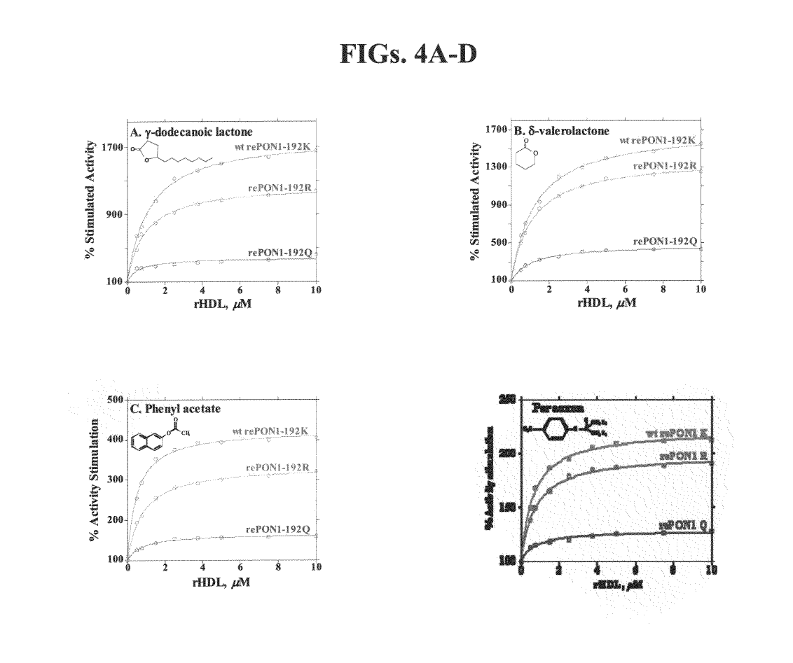

Stimulation of the enzymatic activity was examined by incubating rePON1 isozymes with a range of rHDL concentrations (at the HDL/PON ratio of 0.5-50), and measuring the catalytic activity with various substrates as detailed hereinabove. FIGS. 3A-B illustrate the enzymatic activity of the rePON1 isozymes with δ-nonanoic lactone as substrate (FIG. 3A) and thiobutyl butiryl lactone (TBBL) as substrate (FIG. 3B). FIGS. 4A-D illustrate the enzymatic activity of the rePON1 isozymes with γ-dodecanoic lactone as substrate (FIG. 4A), δ-valerolactone as substrate (FIG. 4B), phenyl acetate as substrate (FIG. 4C) and paraoxon as substrate (FIG. 4D). Data were fitted to the Langmuir saturation curve to give the activation factor Vₘₐₓ (in percent relative to the delipidated PON1) and the apparent affinity Kₐₚₚ. The numbers are displayed in Table 3 hereinbelow.

TABLE 3
Enzymatic activation of rePON1 192 isozymes by apoA-I rHDL

| wt rePON-192 K | | | rePON1-192R | | rePON-192 Q | |
|---|---|---|---|---|---|---|
| Substrate^{a} | Vₘₐₓ (%)^{b} | Kₐₚₚ (μM)^{c} | Vₘₐₓ (%) | Kₐₚₚ (μM) | Vₘₐₓ (%) | Kₐₚₚ (μM) |
| TBBL | 791 ± 10 | 0.5 ± 0.1 | 579 ± 6 | 0.4 ± 0.1 | 340 ± 4 | 0.5 ± 0.1 |
| γ-dodecanoic | 1830 ± 22 | 1.2 ± 0.1 | 1258 ± 21 | 0.9 ± 0.1 | 382 ± 20 | 0.6 ± 0.2 |
| lactone | | | | | | |
| δ-nonanoic | 1580 ± 19 | 1.3 ± 0.1 | 1351 ± 20 | 1.5 ± 0.1 | 778 ± 12 | 0.6 ± 0.1 |
| lactone | | | | | | |
| δ- | 1724 ± 23 | 1.3 ± 0.1 | 1403 ± 13 | 1.1 ± 0.1 | 481 ± 7 | 1.1 ± 0.1 |
| valerolactone | | | | | | |
| phenyl | 425 ± 4 | 0.5 ± 0.1 | 336 ± 4 | 0.8 ± 0.1 | 165 ± 1 | 0.8 ± 0.1 |
| acetate | | | | | | |
| paraoxon | 222 ± 2 | 0.6 ± 0.1 | 200 ± 1 | 0.8 ± 0.1 | 128 ± 1 | 0.8 ± 0.1 |

All values represent the derived parameters with the standard error of the fit.
^{ab}TBBL was taken at 0.25 mM, all other substrates were at 1 mM (≧K_{M} for all the substrates).
^{b}Vₘₐₓ values are presented as the percentage relative to the delipidated enzyme (designated as 100%)
^{c}Kₐₚₚ is the apparent affinity for HDL stimulation.

With all the substrates tested, wt rePON1-192K exhibited the highest stimulation levels, the R isozyme exhibited slightly reduced activation levels (10-30% reduction of Vₘₐₓ), while the stimulation of the Q isozyme was significantly reduced (50-80% reduction of Vₘₐₓ compared to wt rePON1-192K). Taken together with the stability measurements, these results indicate that the K192Q mutation significantly disrupts the ability of rePON1 to associate with HDL, while the K192R substitution preserves the efficient HDL binding properties of the wt rePON1.

### Example 2

### Stability, HDL Binding and Enzymatic Stimulation of Human PON1 Isozymes Bound to rHDL-apoA-I

Materials and Methods

Human PON1-192R and Q isozymes purified from pooled blood samples [Gan, K. N., et al., Drug Metab Dispos, 1991. 19(1): p. 100-6] were kindly provided by Dr. Dragomir Draganov (University of Michigan, Ann Arbour), and stored in presence of 0.1% tergitol and 20% glycerol. Prior to assay, these samples were briefly delipidated [Gaidukov, L. and D. S. Tawfik, Biochemistry, 2005. 44(35): p. 11843-54] and dialyzed against activity buffer to remove the tergitol and glycerol that interfere with HDL binding. Dialyzed samples (0.2 μM) were incubated with rHDL (10 μM). Stability assays were performed at 25° C., in activity buffer supplemented with nitrilotriacetic acid (NTA) and β-mercaptoethanol, both at 5 mM. Data analysis was performed as described above for rePON1s. Stimulation of enzymatic activities was measured with various substrates at 1 mM concentration, except for TBBL (0.25 mM).

Results

The progress of inactivation of human PON1 isozymes anchored on rHDL-apoA-I is illustrated in FIG. 5A. Inactivation of both isozymes followed a two-phase kinetics with similar inactivation rates, but different partitioning between the HDL-bound and unbound phases, as detailed in Table 4 hereinbelow. Amplitudes (A) and kinetic rates of inactivation (k^{inactiv}) were derived by fitting the data to a double-exponential curve.

TABLE 4
Kinetic and equilibrium constants for the inactivation
of human PON1 192 isozymes on apoA-I rHDL

| | A₂(%) | k₁^{inactiv}(hr⁻¹) | k₂^{inactiv} (hr⁻¹) |
|---|---|---|---|
| human PON1-192R | 87 | 0.4 | 0.01 |
| human PON1-192Q | 72 | 0.6 | 0.01 |

Prefixes 1 and 2 designate the first (fast) and the second (slow) phases of the inactivation, respectively. Each value represents the mean of two independent experiments. Standard deviations were less then 10% of parameter values.

The bound phase constituted 87 and 72% for the R and Q isozymes, respectively, indicating the more efficient HDL binding by R192 isozyme.

HDL-mediated stimulation of the enzymatic activity of human PON1 isozymes was determined by incubating PON1 with the highest rHDL-apoA-I concentration (corresponding to the rHDL/PON1 ratio of 50) (FIG. 5B). For all the lactones tested, 192R exhibited about 2-fold higher activation level than 192Q, while the weak stimulation of the promiscuous paraoxonase and arylesterase activities did not differ between the two isozymes. Overall, the large differences in the stability and lactonase activity stimulation of human PON1 isozymes indicate that 192R isozyme interacts more efficiently with HDL than the Q counterpart.

Interestingly, the stimulation levels of human PON1 with most substrates were lower than those determined with rePON1, indicating weaker binding of human PON1 to HDL. This was probably caused by the residual glycerol and tergitol that remained in the protein samples and interfered with HDL binding. In addition, unlike the highly homogeneous preparation of recombinant PON1, PON1 purified from blood samples was shown to contain significant amounts of nonrelevant proteins that copurify with PON1 and can interfere with HDL binding [Morales, R. (2006) Acta Crystallogr D Biol Crystallogr F62, 67-62].

### Example 3

### Correlation of PON1 192R/Q Phenotype with PON1 Stability

Until now, all performed blood tests of PON1 phenotype, status and activity were based on measuring the hydrolysis of paraoxon (or other organophosphates) and aryl esters. However, all these are promiscuous activities of PON1 that are not stimulated by HDL and bare no physiological relevance. Therefore the human sera of individuals bearing either QQ, RQ or RR PON1 phenotypes were tested for their stability and lactonase activity.

Materials and Methods

PON1 Phenotyping in Human Sera: Human sera were collected from 54 healthy individuals at Rambam Medical Center (Haifa, Israel). Sera were divided to aliquots and stored frozen at −20° C. Following thawing, sera were immediately supplemented with β-mercaptoethanol (5 mM) to prevent oxidation, and stored for the duration of the assays at 4° C. (maximum of 1 week). Phenotyping sera for PON1 was performed by a two-substrate method as described [Eckerson, H. W., et al., Am J Hum Genet, 1983. 35(2): p. 214-27]. Briefly, sera were diluted 20-fold in activity buffer, and arylesterase activity was measured in activity buffer containing 1 mM phenyl acetate by monitoring the absorbance at 270 nm (ε=700 OD/M). Paraoxonase activity was measured in buffer containing 50 mM glycine (pH 10.5), 1 mM CaCl₂ and 1 mM paraoxon, either supplemented or not with 1M NaCl, by monitoring the absorbance at 405 nm (ε=11,725 OD/M). The initial rates of product release derived from the two measurements were expressed as U/ml (1 U=1 μmol of phenyl acetate or 1 nmol of paraoxon hydrolyzed per minute per 1 ml of serum). The paraoxonase/arylesterase activity ratio was calculated by dividing the paraoxonase activity of a sample in presence of 1 M NaCl by its arylesterase activity. Stimulation by salt corresponds to the rate of paraoxonase activity in the presence of 1 M NaCl and its absence.

PON1 Inactivation Assays in Human Sera: Sera samples were diluted 10-fold in TBS (10 mM Tris pH 8.0, 150 mM NaCl). Inactivation was initiated by adding an equal volume of inactivation buffer (TBS supplemented with 0.5 mM NTA and 2 mM β-mercaptoethanol) at 25° C. Residual activity was determined with 2 mM phenyl acetate. Inactivation rates fitted well to a mono-exponential fit for all RR sera, and a double-exponential fit was necessary only for RQ and QQ sera. It should be noted that, the reproducibility of these inactivation assays was low. Although the differences between R and Q sera were observed in all assays, the inactivation rates varied from one assay to another. It appears that the sera inactivation kinetics are very sensitive to oxidation. Indeed, supplementing sera with β-mercaptoethanol (5 mM) immediately after defrosting, and storing the β-mercaptoethanol supplemented sera at 4° C. for 12 hrs before the experiment, yielded much more reproducible results.

Lactonase Activity in Human Sera: Lactonase activity was measured in activity buffer containing 0.25 mM TBBL and 0.5 mM DTNB by monitoring the absorbance at 412 nm (ε=7,000 OD/M). Dihydrocoumarin hydrolysis was measured in activity buffer at pH 7.5 by monitoring the absorbance at 270 nm (ε=700 OD/M). Activities were expressed as U/ml, where 1 U of activity is defined as 1 μmol of TBBL or dihydrocoumarin hydrolyzed per minute per 1 ml of serum. The normalized lactonase activity was calculated by dividing TBBLase activity of each sample by its dihydrocoumarin activity.

Antiatherogenic Assays: Delipidated rePON1 isozymes were incubated with a 2.5 or 5-fold molar excess of rHDL-ApoA-I; Cholesterol efflux from macrophages and copper induced oxidation of LDL in presence of HDL-bound rePON1s were performed as described [Rosenblat, M., et al., J Biol Chem, 2006].

Results

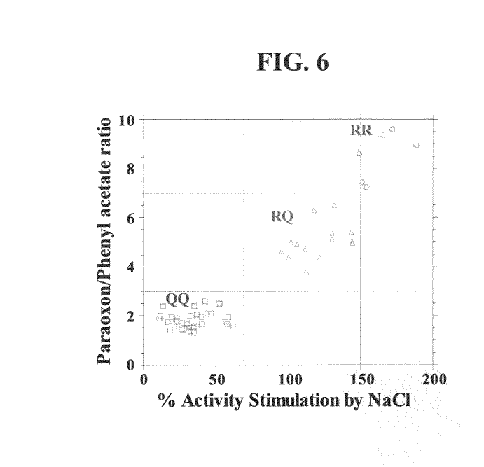

As illustrated in FIG. 6, out of 54 samples, 34 were phenotyped as QQ, 14 as RQ and 6 as RR.

Typical inactivation curves for the selected sera of the three phenotypes are portrayed in FIG. 7A. PON stability differed markedly between the three types of sera. As observed with the reconstituted systems, inactivation of QQ and RQ sera followed the two-phase regime with the fast (1^{st}) and slow (2^{nd}) phases of inactivation, while the RR-type sera followed the mono-exponential decay. The derived inactivation rates for the three types of sera are summarized in Table 5 below.

TABLE 5
Kinetic and equilibrium constants for PON1 inactivation
in human sera of 54 healthy individuals

| | | | | Stability |
|---|---|---|---|---|
| | A₂(% | | | (% residual |
| | slow | K₁^{nactiv} | k₂^{inactiv} | activity af- |
| | phase) | (hr⁻¹) | (hr⁻¹) | ter 9 hours) |
| RR sera (n = 6) | 100 | — | 0.027 ± 0.003 | 79 ± 5 |
| RQ sera (n = 14) | 78 ± 4 | 0.45 ± 0.14 | 0.029 ± 0.005 | 62 ± 4 |
| QQ sera (n = 34) | 67 ± 13 | 0.44 ± 0.15 | 0.043 ± 0.014 | 46 ± 11 |

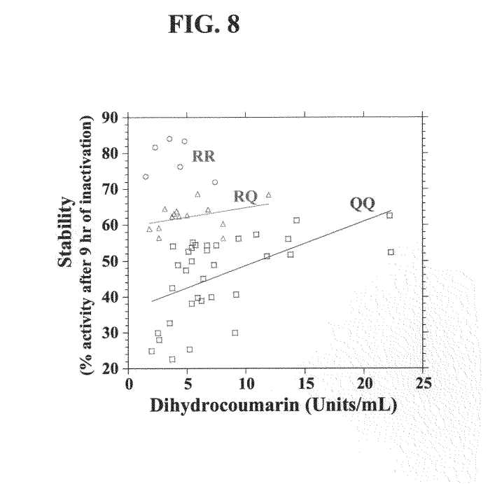

Interestingly, in RQ sera, the inactivation rate of the fast phase was similar to that of the QQ sera, while the rate of the slow phase was similar to the single-phase inactivation rate of RR sera. Thus, in the heterozygote RQ sera, the fast initial phase of the Q isozyme is followed by the slow inactivation of the R isozyme. Marked differences in the stability of the three phenotypes were also observed by measuring PON1's residual activity at a single time point (9 hours; Table 5). Notably, the largest heterogeneity in these values was observed in QQ sera, probably due to large differences in PON1's concentration. Indeed, in QQ sera, there exists a clear correlation between stability and PON1 levels (as determined by levels of dihydrocoumarin activity; see below). It appears that, due to its lower affinity, higher concentrations of PON1-192Q are capable of shifting the equilibrium towards the HDL-bound form, and thereby exhibit increased stability (FIG. 8).

Lactonase activity in human sera was assayed using TBBL that was developed for chromogenic lactonase assays of PON1 [Khersonsky, O. and D. S. Tawfik, Chembiochem, 2006. 7(1): p. 49-53]. Measurements of lactonase activity (expressed as units of TBBLase activity) in 54 human sera are shown in FIG. 9A and Table 6 hereinbelow.

TABLE 6
Lactonase activity in human sera of 54 healthy individuals

| | | | TBBL/ | |
|---|---|---|---|---|
| | TBBL (Units | DHC (Units | DHC | |
| | activity) | activity) | (ratio) | PON1-HDL |
| RR sera (n = 6) | 27.4 ± 13 | 19.9 ± 10.4 | 1.4 ± 0.3 | 22.4 ± 11.7 |
| RQ sera (n = 14) | 20.6 ± 10 | 25.7 ± 14 | 0.8 ± 0.1 | 21.9 ± 11.2 |
| QQ sera (n = 34) | 17.1 ± 8 | 38.1 ± 24.5 | 0.5 ± 0.1 | 26.8 ± 18.5 |

The mean lactonase activity in RR sera was found to be 1.6-fold higher than in QQ sera (27.4 units vs 17.1 units, respectively; FIG. 9A, Table 6). These differences are obviously the combined outcome of two factors: the absolute concentrations of PON1 in the individual sera, and the genotype (R/Q) and consequently the level of stimulation in each serum. To separate these factors, activity levels were tested with dihydrocoumarin. Unlike other lactones, dihydrocoumarin is not stimulated by HDL [Gaidukov, L. and D. S. Tawfik, Biochemistry, 2005. 44(35): p. 11843-54], and the PON1 isozymes hydrolyze it nearly identical rates (at Vₘₐₓ^{Q}=1.125*Vₘₐₓ^{R}). In agreement with previous studies [Humbert, R., et al., Nat Genet, 1993. 3(1): p. 73-6] measurements of dihydrocoumarin hydrolysis in the 54 samples revealed large variations in PON1's concentration (>10-fold), especially in Q-type sera (FIG. 9B). Interestingly, the average dihydrocoumarin activity was found to be almost 2-fold higher in QQ than in RR sera (Table 6). The ratio of TBBL to dihydrocoumarin activity provided the normalized lactonase activity, i.e., the degree of HDL stimulation. Indeed, in agreement with our observations of the reconstituted system, the RR sera exhibit 3-fold higher mean normalized lactonase activity than the QQ sera (FIG. 9C, Table 6).

Antiatherogenic activities of HDL-bound rePON1 isozymes were examined with respect to the stimulation of the HDL-mediated cholesterol efflux from macrophages, and the protection against copper induced LDL oxidation [Rosenblat, M., et al., J Biol Chem, 2006]. The rePON1 isozymes were incubated with rHDL-apoA-I, and added to the cultured macrophages pre-incubated with the labelled cholesterol. The degree of cholesterol efflux was subsequently determined (FIG. 12). The wild-type rePON1-192K increased cholesterol efflux from macrophages (relative to rHDL-apoA-I alone) by 93%, rePON1-192R yielded a slightly milder increase (67%), and rePON1-192Q exhibited the lowest effect (30%). These results correlate well with the levels of HDL binding observed with the three isozymes. Significant differences were not detected in the antioxidation activity of the R and Q isozymes, either in buffer or when bound to HDL-apoA-I. This could be due to technical limitations that do not allow the assay at HDL/PON1 ratios higher than 5:1, whereas complete binding of PON1 to HDL requires ≧50-fold molar excess of HDL.

### CONCLUSIONS

The results of this study unambiguously indicate that the R isozyme binds HDL with higher affinity, and consequently exhibits much higher stability and lipo-lactonase activity, as well as more potent antiatherogenic activity. These differences in HDL-binding, stability and lipo-lactonase are also clearly observed in sera samples obtained from individuals belonging to the QQ, QR and RR genotypes.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications and GenBank Accession numbers mentioned in this specification are herein incorporated in their entirety by reference into the specification, to the same extent as if each individual publication, patent or patent application or GenBank Accession number was specifically and individually indicated to be incorporated herein by reference. In addition, citation or identification of any reference in this application shall not be construed as an admission that such reference is available as prior art to the present invention.

1. A method of determining a stability of a serum PON1-lipoprotein complex, the method comprising measuring an inactivation rate of an enzymatic activity of a PON1 of said PON1-lipoprotein complex, thereby determining the stability of the serum PON1-lipoprotein complex.

2. A method of determining an amount of a stable serum PON1-lipoprotein complex, the method comprising:
(a) determining a fraction of stable serum PON1-lipoprotein complex: total serum PON1-lipoprotein, wherein inactivation of a stable complex follows the kinetics of a second phase of a double-exponential inactivation plot; and
(b) determining a total level of serum PON1, wherein said fraction multiplied by said total level of serum PON1 is the amount of stable serum PON1-lipoprotein complex.

3. The method of claim 2, wherein step (a)
is effected following inactivation with an inactivator for a predetermined time.

4. A method of determining a normalized lactonase activity of serum PON1, the method comprising determining in a sample of a subject:
(a) a lactonase activity of serum PON1; and
(b) a total level of serum PON1, whereby a ratio of said lactonase activity: said total level is the normalized lactonase activity of serum PON1.

5. A method of diagnosing a lipid-related disorder, the method comprising determining in a sample of a subject a normalized lactonase activity of PON1, thereby diagnosing the lipid-related disorder.

6. A method of diagnosing a lipid-related disorder, the method comprising determining in a sample of a subject a fraction of stable serum PON1-lipoprotein complex: total PON1-lipoprotein complex, thereby diagnosing the lipid-related disorder.

7-9. (canceled)

10. The method of claim 1, wherein said PON1-lipoprotein complex comprises HDL-apoA-I.

11. The method of claim 5, wherein said lipid-related disorder is selected from the group consisting of a cardiovascular disorder,
a pancreatic disorder and a neurological disorder.

12-20. (canceled)

21. The method of claim 6, further comprising determining a lactonase activity of serum PON1.

22. The method of claim 5, further comprising determining in a sample of said subject a fraction of stable PON1-lipoprotein complex: total PON1-lipoprotein complex.

23. The method of claim 6, further comprising determining in a sample of said subject an amount of total serum PON1.

24-25. (canceled)

26. The method of claim 21 wherein said lactonase activity is a normalized lactonase activity.

27. (canceled)

28. The method of claim 4, wherein said determining said lactonase activity of serum PON1 is effected using 5-(thiobutyl)-butyrolactone (TBBL).

29. (canceled)

30. The method of claim 6, wherein said determining an amount of a stable serum PON1-lipoprotein complex is effected by
(a) determining a fraction of stable serum PON1-lipoprotein complex: total serum PON1-lipoprotein complex; and
(b) determining a total level of serum PON1, wherein said fraction multiplied by said total level of serum PON1 is the amount of stable serum PON1-lipoprotein complex.

31. The method of claim 2, wherein said determining said fraction of stable serum PON1-lipoprotein complex is effected by measuring an inactivation rate of an enzymatic activity of a PON1 of said PON1-lipoprotein complex.

32. The method of claim 1, wherein said measuring an inactivation rate is effected using a PON1 inactivator.

33-35. (canceled)

36. The method of claim 3, wherein said PON1 inactivator is NTA.

37-41. (canceled)

42. The method of claim 2, wherein said PON1-lipoprotein complex comprises HDL-apoA-I.

43. The method of claim 6, wherein said lipid-related disorder is selected from the group consisting of a cardiovascular disorder, a pancreatic disorder and a neurological disorder.

44. The method of claim 21, wherein said determining said lactonase activity of serum PON1 is effected using 5-(thiobutyl)-butyrolactone (TBBL).

45. The method of claim 31, wherein said measuring an inactivation rate is effected using a PON1 inactivator.

46. The method of claim 32, wherein said PON1 inactivator is NTA.

47. The method of claim 30, wherein said determining said fraction of stable serum PON1-lipoprotein complex is effected by measuring an inactivation rate of an enzymatic activity of a PON1 of said PON1-lipoprotein complex.


SEQUENCE LISTING (16 sequences)

SEQ ID NO: 1 - DNA, 1065 bp, Artificial sequence
  WT rePON1-192K
atggctaaactgacagcgctcacactcttggggctgggattggcactcttcgatggacagaagtcttctttccaaacacgatttaatgttcaccgtgaagtaactccagtggaacttcctaactgtaatttagttaaaggggttgacaatggttctgaagacttggaaatactgcccaatggactggctttcatcagctccggattaaagtatcctggaataatgagctttgaccctgataagtctggaaagatacttctaatggacctgaatgaggaagacccagtagtgttggaactgggcattactggaaatacattggatatatcttcatttaaccctcatgggattagcacattcacagatgaagataacactgtgtacctactggtggtaaaccatccagactcctcgtccaccgtggaggtgtttaaatttcaagaagaagaaaaatcacttttgcatctgaaaaccatcagacacaagcttctgcctagtgtgaatgacattgtcgctgtgggacctgaacacttttatgccacaaatgatcactattttgctgacccttacttaaaatcctgggaaatgcatttgggattagcgtggtcatttgttacttattatagtcccaatgatgttcgagtagtggcagaaggatttgattttgctaacggaatcaacatctcaccagacggcaagtatgtctatatagctgagttgctggctcataagatccatgtgtatgaaaagcacgctaattggactttaactccattgaagtccctcgactttgacacccttgtggataacatctctgtggatcctgtgacaggggacctctgggtgggatgccatcccaacggaatgcgaatcttctactatgacccaaagaatcctcccggctcagaggtgcttcgaatccaggacattttatccgaagagcccaaagtgacagtggtttatgcagaaaatggcactgtgttacagggcagcacggtggccgctgtgtacaaagggaaactgctgattggcacagtgtttcacaaagctctttactgtgagctg

SEQ ID NO: 2 - protein, 355 aa, Artificial sequence
  WT rePON1-192K
MAKLTALTLLGLGLALFDGQKSSFQTRFNVHREVTPVELPNCNLVKGVDNGSEDLEILPNGLAFISSGLKYPGIMSFDPDKSGKILLMDLNEEDPVVLELGITGNTLDISSFNPHGISTFTDEDNTVYLLVVNHPDSSSTVEVFKFQEEEKSLLHLKTIRHKLLPSVNDIVAVGPEHFYATNDHYFADPYLKSWEMHLGLAWSFVTYYSPNDVRVVAEGFDFANGINISPDGKYVYIAELLAHKIHVYEKHANWTLTPLKSLDFDTLVDNISVDPVTGDLWVGCHPNGMRIFYYDPKNPPGSEVLRIQDILSEEPKVTVVYAENGTVLQGSTVAAVYKGKLLIGTVFHKALYCEL

SEQ ID NO: 3 - DNA, 1065 bp, Artificial sequence
  rePON1-192R isozyme
atggctaaactgacagcgctcacactcttggggctgggattggcactcttcgatggacagaagtcttctttccaaacacgatttaatgttcaccgtgaagtaactccagtggaacttcctaactgtaatttagttaaaggggttgacaatggttctgaagacttggaaatactgcccaatggactggctttcatcagctccggattaaagtatcctggaataatgagctttgaccctgataagtctggaaagatacttctaatggacctgaatgaggaagacccagtagtgttggaactgggcattactggaaatacattggatatatcttcatttaaccctcatgggattagcacattcacagatgaagataacactgtgtacctactggtggtaaaccatccagactcctcgtccaccgtggaggtgtttaaatttcaagaagaagaaaaatcacttttgcatctgaaaaccatcagacacaagcttctgcctagtgtgaatgacattgtcgctgtgggacctgaacacttttatgccacaaatgatcactattttgctgacccttacttacgttcctgggaaatgcatttgggattagcgtggtcatttgttacttattatagtcccaatgatgttcgagtagtggcagaaggatttgattttgctaacggaatcaacatctcaccagacggcaagtatgtctatatagctgagttgctggctcataagatccatgtgtatgaaaagcacgctaattggactttaactccattgaagtccctcgactttgacacccttgtggataacatctctgtggatcctgtgacaggggacctctgggtgggatgccatcccaacggaatgcgaatcttctactatgacccaaagaatcctcccggctcagaggtgcttcgaatccaggacattttatccgaagagcccaaagtgacagtggtttatgcagaaaatggcactgtgttacagggcagcacggtggccgctgtgtacaaagggaaactgctgattggcacagtgtttcacaaagctctttactgtgagctg

SEQ ID NO: 4 - protein, 355 aa, Artificial sequence
  rePON1-192R isozyme
MAKLTALTLLGLGLALFDGQKSSFQTRFNVHREVTPVELPNCNLVKGVDNGSEDLEILPNGLAFISSGLKYPGIMSFDPDKSGKILLMDLNEEDPVVLELGITGNTLDISSFNPHGISTFTDEDNTVYLLVVNHPDSSSTVEVFKFQEEEKSLLHLKTIRHKLLPSVNDIVAVGPEHFYATNDHYFADPYLRSWEMHLGLAWSFVTYYSPNDVRVVAEGFDFANGINISPDGKYVYIAELLAHKIHVYEKHANWTLTPLKSLDFDTLVDNISVDPVTGDLWVGCHPNGMRIFYYDPKNPPGSEVLRIQDILSEEPKVTVVYAENGTVLQGSTVAAVYKGKLLIGTVFHKALYCEL

SEQ ID NO: 5 - DNA, 1065 bp, Artificial sequence
  rePON1-192Q isozyme
atggctaaactgacagcgctcacactcttggggctgggattggcactcttcgatggacagaagtcttctttccaaacacgatttaatgttcaccgtgaagtaactccagtggaacttcctaactgtaatttagttaaaggggttgacaatggttctgaagacttggaaatactgcccaatggactggctttcatcagctccggattaaagtatcctggaataatgagctttgaccctgataagtctggaaagatacttctaatggacctgaatgaggaagacccagtagtgttggaactgggcattactggaaatacattggatatatcttcatttaaccctcatgggattagcacattcacagatgaagataacactgtgtacctactggtggtaaaccatccagactcctcgtccaccgtggaggtgtttaaatttcaagaagaagaaaaatcacttttgcatctgaaaaccatcagacacaagcttctgcctagtgtgaatgacattgtcgctgtgggacctgaacacttttatgccacaaatgatcactattttgctgacccttacttacagtcctgggaaatgcatttgggattagcgtggtcatttgttacttattatagtcccaatgatgttcgagtagtggcagaaggatttgattttgctaacggaatcaacatctcaccagacggcaagtatgtctatatagctgagttgctggctcataagatccatgtgtatgaaaagcacgctaattggactttaactccattgaagtccctcgactttgacacccttgtggataacatctctgtggatcctgtgacaggggacctctgggtgggatgccatcccaacggaatgcgaatcttctactatgacccaaagaatcctcccggctcagaggtgcttcgaatccaggacattttatccgaagagcccaaagtgacagtggtttatgcagaaaatggcactgtgttacagggcagcacggtggccgctgtgtacaaagggaaactgctgattggcacagtgtttcacaaagctctttactgtgagctg

SEQ ID NO: 6 - protein, 355 aa, Artificial sequence
  rePON1-192Q isozyme
MAKLTALTLLGLGLALFDGQKSSFQTRFNVHREVTPVELPNCNLVKGVDNGSEDLEILPNGLAFISSGLKYPGIMSFDPDKSGKILLMDLNEEDPVVLELGITGNTLDISSFNPHGISTFTDEDNTVYLLVVNHPDSSSTVEVFKFQEEEKSLLHLKTIRHKLLPSVNDIVAVGPEHFYATNDHYFADPYLQSWEMHLGLAWSFVTYYSPNDVRVVAEGFDFANGINISPDGKYVYIAELLAHKIHVYEKHANWTLTPLKSLDFDTLVDNISVDPVTGDLWVGCHPNGMRIFYYDPKNPPGSEVLRIQDILSEEPKVTVVYAENGTVLQGSTVAAVYKGKLLIGTVFHKALYCEL

SEQ ID NO: 7 - DNA, 1011 bp, Artificial sequence
  Delta20-rePON1-192K isozyme
atggcgaagtcttctttccaaacacgatttaatgttcaccgtgaagtaactccagtggaacttcctaactgtaatttagttaaaggggttgacaatggttctgaagacttggaaatactgcccaatggactggctttcatcagctccggattaaagtatcctggaataatgagctttgaccctgataagtctggaaagatacttctaatggacctgaatgaggaagacccagtagtgttggaactgggcattactggaaatacattggatatatcttcatttaaccctcatgggattagcacattcacagatgaagataacactgtgtacctactggtggtaaaccatccagactcctcgtccaccgtggaggtgtttaaatttcaagaagaagaaaaatcacttttgcatctgaaaaccatcagacacaagcttctgcctagtgtgaatgacattgtcgctgtgggacctgaacacttttatgccacaaatgatcactattttgctgacccttacttaaaatcctgggaaatgcatttgggattagcgtggtcatttgttacttattatagtcccaatgatgttcgagtagtggcagaaggatttgattttgctaacggaatcaacatctcaccagacggcaagtatgtctatatagctgagttgctggctcataagatccatgtgtatgaaaagcacgctaattggactttaactccattgaagtccctcgactttgacacccttgtggataacatctctgtggatcctgtgacaggggacctctgggtgggatgccatcccaacggaatgcgaatcttctactatgacccaaagaatcctcccggctcagaggtgcttcgaatccaggacattttatccgaagagcccaaagtgacagtggtttatgcagaaaatggcactgtgttacagggcagcacggtggccgctgtgtacaaagggaaactgctgattggcacagtgtttcacaaagctctttactgtgagctg

SEQ ID NO: 8 - protein, 337 aa, Artificial sequence
  Delta20-rePON1-192K isozyme
MAKSSFQTRFNVHREVTPVELPNCNLVKGVDNGSEDLEILPNGLAFISSGLKYPGIMSFDPDKSGKILLMDLNEEDPVVLELGITGNTLDISSFNPHGISTFTDEDNTVYLLVVNHPDSSSTVEVFKFQEEEKSLLHLKTIRHKLLPSVNDIVAVGPEHFYATNDHYFADPYLKSWEMHLGLAWSFVTYYSPNDVRVVAEGFDFANGINISPDGKYVYIAELLAHKIHVYEKHANWTLTPLKSLDFDTLVDNISVDPVTGDLWVGCHPNGMRIFYYDPKNPPGSEVLRIQDILSEEPKVTVVYAENGTVLQGSTVAAVYKGKLLIGTVFHKALYCEL

SEQ ID NO: 9 - DNA, 1011 bp, Artificial sequence
  Delta20-rePON1-192R isozyme
atggcgaagtcttctttccaaacacgatttaatgttcaccgtgaagtaactccagtggaacttcctaactgtaatttagttaaaggggttgacaatggttctgaagacttggaaatactgcccaatggactggctttcatcagctccggattaaagtatcctggaataatgagctttgaccctgataagtctggaaagatacttctaatggacctgaatgaggaagacccagtagtgttggaactgggcattactggaaatacattggatatatcttcatttaaccctcatgggattagcacattcacagatgaagataacactgtgtacctactggtggtaaaccatccagactcctcgtccaccgtggaggtgtttaaatttcaagaagaagaaaaatcacttttgcatctgaaaaccatcagacacaagcttctgcctagtgtgaatgacattgtcgctgtgggacctgaacacttttatgccacaaatgatcactattttgctgacccttacttacgttcctgggaaatgcatttgggattagcgtggtcatttgttacttattatagtcccaatgatgttcgagtagtggcagaaggatttgattttgctaacggaatcaacatctcaccagacggcaagtatgtctatatagctgagttgctggctcataagatccatgtgtatgaaaagcacgctaattggactttaactccattgaagtccctcgactttgacacccttgtggataacatctctgtggatcctgtgacaggggacctctgggtgggatgccatcccaacggaatgcgaatcttctactatgacccaaagaatcctcccggctcagaggtgcttcgaatccaggacattttatccgaagagcccaaagtgacagtggtttatgcagaaaatggcactgtgttacagggcagcacggtggccgctgtgtacaaagggaaactgctgattggcacagtgtttcacaaagctctttactgtgagctg

SEQ ID NO: 10 - protein, 337 aa, Artificial sequence
  Delta20-rePON1-192R isozyme
MAKSSFQTRFNVHREVTPVELPNCNLVKGVDNGSEDLEILPNGLAFISSGLKYPGIMSFDPDKSGKILLMDLNEEDPVVLELGITGNTLDISSFNPHGISTFTDEDNTVYLLVVNHPDSSSTVEVFKFQEEEKSLLHLKTIRHKLLPSVNDIVAVGPEHFYATNDHYFADPYLRSWEMHLGLAWSFVTYYSPNDVRVVAEGFDFANGINISPDGKYVYIAELLAHKIHVYEKHANWTLTPLKSLDFDTLVDNISVDPVTGDLWVGCHPNGMRIFYYDPKNPPGSEVLRIQDILSEEPKVTVVYAENGTVLQGSTVAAVYKGKLLIGTVFHKALYCEL

SEQ ID NO: 11 - DNA, 1011 bp, Artificial sequence
  Delta20-rePON1-192Q isozyme
atggcgaagtcttctttccaaacacgatttaatgttcaccgtgaagtaactccagtggaacttcctaactgtaatttagttaaaggggttgacaatggttctgaagacttggaaatactgcccaatggactggctttcatcagctccggattaaagtatcctggaataatgagctttgaccctgataagtctggaaagatacttctaatggacctgaatgaggaagacccagtagtgttggaactgggcattactggaaatacattggatatatcttcatttaaccctcatgggattagcacattcacagatgaagataacactgtgtacctactggtggtaaaccatccagactcctcgtccaccgtggaggtgtttaaatttcaagaagaagaaaaatcacttttgcatctgaaaaccatcagacacaagcttctgcctagtgtgaatgacattgtcgctgtgggacctgaacacttttatgccacaaatgatcactattttgctgacccttacttacagtcctgggaaatgcatttgggattagcgtggtcatttgttacttattatagtcccaatgatgttcgagtagtggcagaaggatttgattttgctaacggaatcaacatctcaccagacggcaagtatgtctatatagctgagttgctggctcataagatccatgtgtatgaaaagcacgctaattggactttaactccattgaagtccctcgactttgacacccttgtggataacatctctgtggatcctgtgacaggggacctctgggtgggatgccatcccaacggaatgcgaatcttctactatgacccaaagaatcctcccggctcagaggtgcttcgaatccaggacattttatccgaagagcccaaagtgacagtggtttatgcagaaaatggcactgtgttacagggcagcacggtggccgctgtgtacaaagggaaactgctgattggcacagtgtttcacaaagctctttactgtgagctg

SEQ ID NO: 12 - protein, 337 aa, Artificial sequence
  Delta20-rePON1-192Q isozyme
MAKSSFQTRFNVHREVTPVELPNCNLVKGVDNGSEDLEILPNGLAFISSGLKYPGIMSFDPDKSGKILLMDLNEEDPVVLELGITGNTLDISSFNPHGISTFTDEDNTVYLLVVNHPDSSSTVEVFKFQEEEKSLLHLKTIRHKLLPSVNDIVAVGPEHFYATNDHYFADPYLQSWEMHLGLAWSFVTYYSPNDVRVVAEGFDFANGINISPDGKYVYIAELLAHKIHVYEKHANWTLTPLKSLDFDTLVDNISVDPVTGDLWVGCHPNGMRIFYYDPKNPPGSEVLRIQDILSEEPKVTVVYAENGTVLQGSTVAAVYKGKLLIGTVFHKALYCEL

SEQ ID NO: 13 - DNA, 1065 bp, Homo sapiens
  misc_feature: 192R variant
atggcgaagctgattgcgctcaccctcttggggatgggactggcactcttcaggaaccaccagtcttcttaccaaacacgacttaatgctctccgagaggtacaacccgtagaacttcctaactgtaatttagttaaaggaatcgaaactggctctgaagacttggagatactgcctaatggactggctttcattagctctggattaaagtatcctggaataaagagcttcaaccccaacagtcctggaaaaatacttctgatggacctgaatgaagaagatccaacagtgttggaattggggatcactggaagtaaatttgatgtatcttcatttaaccctcatgggattagcacattcacagatgaagataatgccatgtacctcctggtggtgaaccatccagatgccaagtccacagtggagttgtttaaatttcaagaagaagaaaaatcgcttttgcatctaaaaaccatcagacataaacttctgcctaatttgaatgatattgttgctgtgggacctgagcacttttatggcacaaatgatcactattttcttgacccctacttacgatcctgggagatgtatttgggtttagcgtggtcgtatgttgtctactatagtccaagtgaagttcgagtggtggcagaaggatttgattttgctaatggaatcaacatttcacccgatggcaagtatgtctatatagctgagttgctggctcataagattcatgtgtatgaaaagcatgctaattggactttaactccattgaagtcccttgactttaataccctcgtggataacatatctgtggatcctgagacaggagacctttgggttggatgccatcccaatggcatgaaaatcttcttctatgactcagagaatcctcctgcatcagaggtgcttcgaatccagaacattctaacagaagaacctaaagtgacacaggtttatgcagaaaatggcacagtgttgcaaggcagtacagttgcctctgtgtacaaagggaaactgctgattggcacagtgtttcacaaagctctttactgtgagctc

SEQ ID NO: 14 - protein, 355 aa, Homo sapiens [sequence omitted]

SEQ ID NO: 15 - DNA, 1065 bp, Homo sapiens
  misc_feature: 192Q variant
atggcgaagctgattgcgctcaccctcttggggatgggactggcactcttcaggaaccaccagtcttcttaccaaacacgacttaatgctctccgagaggtacaacccgtagaacttcctaactgtaatttagttaaaggaatcgaaactggctctgaagacttggagatactgcctaatggactggctttcattagctctggattaaagtatcctggaataaagagcttcaaccccaacagtcctggaaaaatacttctgatggacctgaatgaagaagatccaacagtgttggaattggggatcactggaagtaaatttgatgtatcttcatttaaccctcatgggattagcacattcacagatgaagataatgccatgtacctcctggtggtgaaccatccagatgccaagtccacagtggagttgtttaaatttcaagaagaagaaaaatcgcttttgcatctaaaaaccatcagacataaacttctgcctaatttgaatgatattgttgctgtgggacctgagcacttttatggcacaaatgatcactattttcttgacccctacttacaatcctgggagatgtatttgggtttagcgtggtcgtatgttgtctactatagtccaagtgaagttcgagtggtggcagaaggatttgattttgctaatggaatcaacatttcacccgatggcaagtatgtctatatagctgagttgctggctcataagattcatgtgtatgaaaagcatgctaattggactttaactccattgaagtcccttgactttaataccctcgtggataacatatctgtggatcctgagacaggagacctttgggttggatgccatcccaatggcatgaaaatcttcttctatgactcagagaatcctcctgcatcagaggtgcttcgaatccagaacattctaacagaagaacctaaagtgacacaggtttatgcagaaaatggcacagtgttgcaaggcagtacagttgcctctgtgtacaaagggaaactgctgattggcacagtgtttcacaaagctctttactgtgagctc

SEQ ID NO: 16 - protein, 355 aa, Homo sapiens [sequence omitted]